(19) United States
(12) Patent Application Publication
(10) Pub. No.: US 2026/0144478 A1
(43) Pub. Date: May 28, 2026

(54) METHOD AND SYSTEM FOR FETAL MOVEMENT MONITORING

(71) Applicant: Baymatob Pty Ltd, Sydney (AU)

(72) Inventors: Sarah Catherine McDonald, Sydney (AU); Rishi Ramakrishnan, Sydney (AU)

(73) Assignee: Baymatob Pty Ltd, Sydney (AU)

(21) Appl. No.: 19/119,908

(22) PCT Filed: Oct. 10, 2023
(86) PCT No.: PCT/AU2023/050999

§ 371 Date: Apr. 10, 2025

(30) Foreign Application Priority Data
Oct. 10, 2022 (AU) 2022902956

(51) Int. Cl. A61B 5/00; A61B 5/296; G16H 40/63

(52) U.S. Cl. CPC A61B 5/4362; A61B 5/296; A61B 5/7264; A61B 5/742; G16H 40/63; A61B 2562/0219

(57) ABSTRACT

Disclosed is a method of determining fetal movement during pregnancy or labour. The method comprises receiving patient data from measurements from a first sensor and a second sensor attached to a patient, each of the first and second sensors being selected from a set of sensor types consisting of an EMG (Electromyography) sensor, a flex sensor and an accelerometer, the patient data from the second sensor allowing detection of fetal movement when physiological signals impede detection of the fetal movement in the patient data from the first sensor, wherein the first sensor is a different sensor type from the second sensor. The method also comprises determining that fetal movement has occurred by processing the received patient data from the first and second sensors in order to indicate the determined fetal movement.

### RELATED APPLICATION

This application claims priority from Australian Provisional Patent Application No. 2022902956, the entire contents of which are incorporated herein by reference.

### TECHNICAL FIELD

The present disclosure relates to detecting fetal movement.

### BACKGROUND

Fetal movement detection and measurement is an important measure to assess fetal wellbeing in utero and to help determine whether complications may be present. Fetal movement detection and measurement may also determine whether interventions are required.

There are a number of outcomes associated with a decrease in fetal movement during pregnancy with perhaps the most publicised being that of stillbirth. One problem with relying on fetal movement detection and measurement is that current methods of identifying and assessing fetal movement are subjective and rely on the perception of a pregnant individual. This can be problematic for two reasons. Firstly, cases of concern may be missed and/or identified too late and secondly, cases may be incorrectly identified as problematic and an intervention made unnecessarily. Further, subjective methods may provide unreliable information. For example, identifying normal fetal movement patterns as well as defining normal fetal movement to a level that allows for advancements towards earlier detection of abnormalities with the hope of improving outcomes while also reducing rates of unnecessary intervention may be difficult.

Current methods of clinically assessing fetal movement include both in hospital and at home methods. In a hospital setting, pregnant individuals are attached to electronic fetal monitoring (EFM) and provided a button linked with the EFM to push at when they feel movement. A mark is placed on the EFM trace at the location of the movement, for clinical reference with respect to the EFM trace, allowing clinicians to assess reported movements alongside fetal heartrate and uterine activity.

In a home setting, pregnant individuals may be asked to monitor fetal movements regularly of their own accord. This method is known as kick counting. Kick counting is usually recommended in the third trimester of pregnancy and involves the pregnant individual sitting or lying for a period of time and focusing on fetal movements they feel during a pre-defined time period, with fetal movements totalled.

While these assessments are better than nothing, their shortcomings come in both the subjectivity and the requirement for maternal consciousness. With respect to subjectivity, research has shown that results may vary wildly between reported movement and actual movement, with correlation of 37-88% between maternal perception of fetal movement and fetal movement detected by ultrasound.

Another drawback of self-reported movement measurement is that information may only be collected when the pregnant individual is awake. This means movement cannot be measured when the pregnant individual is asleep, so measurement of fetal movement may not be possible during problematic or higher risk times, such as during sleep where the maternal sleep position may restrict blood flow.

Some EMF devices have attempted to automate the process of identifying fetal movements with little success and/or reliability resulting in maternal perception remaining the standard of care to date.

It is desired to address or ameliorate one or more disadvantages or limitations associated with the prior art, or to at least provide a useful alternative.

### SUMMARY

This Summary is provided to introduce a selection of concepts in a simplified form that are further described below in the Detailed Description. This Summary is not intended to identify essential features of the claimed subject matter, nor is it intended to be used to limit the scope of the claimed subject matter.

Disclosed is a method of determining fetal movement during pregnancy or labour, the method comprising: receiving patient data from measurements from a first sensor and a second sensor attached to a patient, each of the first and second sensors being selected from a set of sensor types consisting of an EMG (Electromyography) sensor, a flex sensor and an accelerometer, the patient data from the second sensor allowing detection of fetal movement when physiological signals impede detection of the fetal movement in the patient data from the first sensor, wherein the first sensor is a different sensor type from the second sensor; and determining that fetal movement has occurred by processing the received patient data from the first and second sensors in order to indicate the determined fetal movement.

In one embodiment, the patient data from the first sensor and the second sensor are processed separately to determine that fetal movement has occurred and the determination of fetal movement based on the first sensor and the second sensor are combined to determine fetal movement.

In one embodiment, the patient data from the first sensor and the second sensor are used as input to a trained classifier to determine that fetal movement has occurred.

In one embodiment, the patient data from the first sensor and the second sensor are used to generate a feature vector that is input to the trained classifier.

In one embodiment, processing the received patient data to determine a measure of fetal movement comprises applying a sliding window to the patient data.

In one embodiment, the first sensor is an accelerometer and processing the patient data to determine a measure of fetal movement comprises determining a displacement.

In one embodiment, the method further comprises: receiving maternal input of fetal movement perception and comparing the maternal input with the determined fetal movement.

In one embodiment, determining the fetal movement uses the maternal presentation information.

In one embodiment, the indication of the determined fetal movement includes a time based display showing a time when the fetal movement was detected.

In one embodiment, the indication of the determined fetal movement includes a location based display providing an indication where on the patient the fetal movement was detected.

In one embodiment, the first sensor is an EMG sensor attached to the patient and determining the location of the fetal movement comprises: determining electrode pair vectors between individual electrodes of the EMG sensor; selecting two of the electrode pair vectors having a similar signal; determining the location of the fetal movement by selecting an electrode from the electrodes of the EMG sensor, the selected electrode being common to the selected two electrode pair vectors and positioned at the determined location.

In one embodiment, one of the electrode is a reference electrode.

Also disclosed is a method of determining fetal movement during pregnancy or labour, the method comprising: receiving patient data from measurements from a first sensor and a second sensor attached to a patient, each of the first and second sensors being selected from the set of sensor types consisting of an EMG (Electromyography) sensor, a flex sensor and an accelerometer; and determining a type of fetal movement that occurred by processing the received patient data from the first and second sensors in order to indicate the type of fetal movement, the patient data from the first sensor allowing determination of a type of fetal movement in combination with the patient data from the second sensor.

In one embodiment, the first sensor is a different sensor type from the second sensor.

In one embodiment, the fetal movement type is determined according to an intensity of the fetal movement.

In one embodiment, the intensity of the fetal movement is determined based on a location of the fetal movement.

In one embodiment, the first sensor is an EMG sensor attached to the patient and determining the location of the fetal movement comprises: determining electrode pair vectors between individual electrodes of the EMG sensor; selecting two of the electrode pair vectors having a similar signal; determining the location of the fetal movement by selecting an electrode from the electrodes of the EMG sensor, the selected electrode being common to the selected two electrode pair vectors and positioned at the determined location.

In one embodiment, fetal movement is determined to occur at a second electrode of the electrodes of the EMG sensor, based on the electrode pair vectors, the second electrode being attached to the patient at a second location.

In one embodiment, the fetal movement type is determined based on the location and the second location.

In one embodiment, the second sensor is a flex senor and a location of the flex sensor are used to determine the location of the fetal movement.

In one embodiment, one of the first and second sensors is an accelerometer.

In one embodiment, the patient data from the accelerometer is acceleration data allowing differentiation of the intensity of the fetal movement between small and large movement.

In one embodiment, the intensity of the fetal movement is determined according to the combination of the patient data from the first and the second sensors.

In one embodiment, the type of fetal movement is determined based on a location of the fetal movement.

In one embodiment, respective sensor types of the first sensor and the second sensor are used to determine the fetal movement type.

In one embodiment, one of the electrode is a reference electrode.

In one embodiment, the method further comprises: receiving maternal input of fetal movement perception and comparing the maternal input with the determined fetal movement.

In one embodiment, determining the fetal movement uses the maternal presentation information.

In one embodiment, the indicating of the type of fetal movement includes a time based display showing a time when the fetal movement was detected.

In one embodiment, the indicating of the type of fetal movement includes a location based display providing an indication where on the patient the fetal movement was detected.

In one embodiment, a system is configured to perform the method as set out above

Also disclosed is a method for determining fetal movement during pregnancy or labour, the method comprising: receiving patient data from measurements from a sensor attached to a patient, the sensor being selected from the set consisting of an EMG sensor, a flex sensor and an accelerometer; and determining a type of fetal movement that occurred by processing the received patient data from the sensor in order to indicate the type of fetal movement.

### BRIEF DESCRIPTION OF DRAWINGS

At least one embodiment of the present invention is described, by way of example only, with reference to the accompanying drawings, in which:

FIG. 1 illustrates a functional block diagram of an example processing system that can be utilised to embody or give effect to a particular embodiment;

FIG. 2 illustrates an example network infrastructure that can be utilised to embody or give effect to a particular embodiment;

FIG. 3 illustrates a flowchart of the use of a fetal movement monitoring device to detect fetal movement;

FIG. 4 illustrates an example of an electrode assembly;

FIG. 5 illustrates an example of a medical device that can be used together with the electrode assembly of FIG. 4;

FIGS. 6A and 6B illustrate an exemplary method of using the electrode assembly;

FIG. 7 illustrates a device station for the medical device of FIG. 5;

FIG. 8 illustrates processing patient data for a fetal movement monitoring process;

FIG. 9 illustrates an EMG fetal movement monitoring process;

FIG. 10 illustrates a flex sensor fetal movement monitoring process;

FIG. 11 illustrates an accelerometer fetal movement monitoring process;

FIG. 12 illustrates a multi-sensor fetal movement monitoring process;

FIG. 13 illustrates an alternative multi-sensor fetal movement monitoring process;

FIG. 14 illustrates a fetal movement monitoring system;

FIGS. 15A and 15B illustrate an example of a medical device; and

FIG. 16A shows an example of a medical device;

FIGS. 16B and 16C show combined sensor data outputs from the medical device of FIG. 16A;

FIG. 16D shows an example of a medical device;

FIG. 17 illustrates a maternal presentation information collection system;

FIG. 18 illustrates a manual movement marking process;

FIGS. 19A, 19B and 19C show representations of fetal movement; and

FIGS. 20A, 20B, 20C, 20D and 20E show representations of fetal movement.

### DETAILED DESCRIPTION

The following modes, given by way of example only, are described in order to provide a more precise understanding of one or more embodiments. In the drawings, like reference numerals are used to identify like parts throughout the drawings.

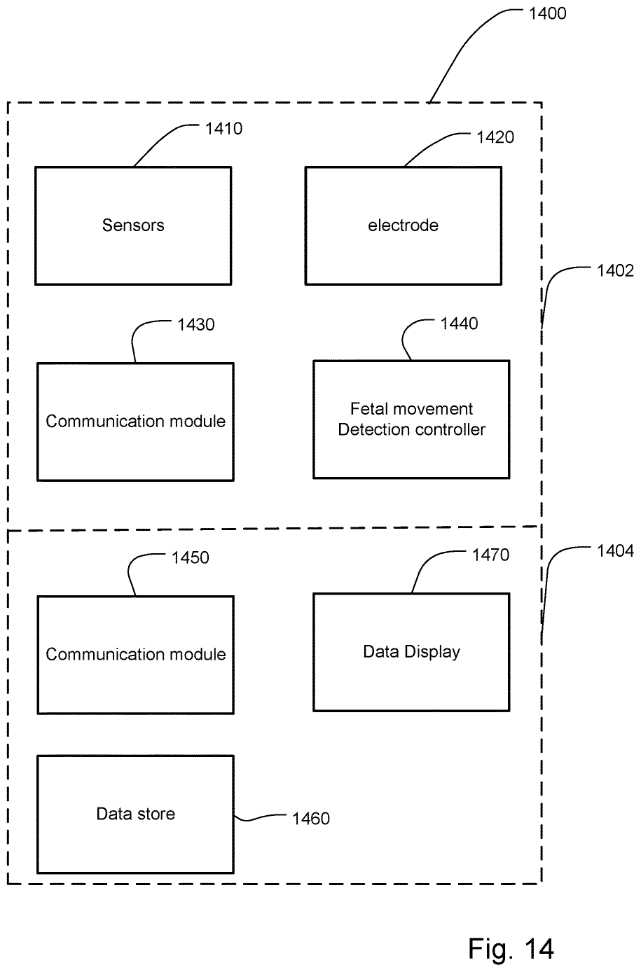

FIG. 14 shows a fetal movement monitoring system 1400 according to at least one embodiment of the present disclosure that may be used to detect fetal movement. The fetal movement monitoring system 1400 has two components, a fetal movement monitoring device 1402 and a fetal movement detection controller 1404 in communication with the fetal movement monitoring device 1402. The Fetal movement detection controller 1404 operates as a supervisory system to collect, store and display data communicated from the fetal movement monitoring device 1402

The fetal movement monitoring device 1402 has one or more sensors 1410. The sensors may be one or more of an electrical potential sensor, a movement sensor, a deformation sensor, a temperature sensor, and/or a patient response sensor. Some of the sensors 1410, such as an electrical potential sensor, may be attached to one or more electrodes 1420 for attaching the one or more sensors 1410 to a body of the patient. The operation of the fetal movement monitoring device 1402 is controlled locally by a device controller 1440.

The one or more sensors 1410 collect patient data that may be transmitted by a communication module 1430 to the fetal movement detection controller 1404. In one example, components of the fetal movement monitoring system 1400, including the sensors 1410, electrodes 1420, the communication module 1430, and the device controller 1440 may be located in a housing (thus forming a monitoring, or medical, device), e.g., a water proof and impact resistant housing. Some components of the fetal movement monitoring system 1400 may be located separately to the housing such as the fetal movement detection controller 1404. In one example, components of the fetal movement detection controller 1404 may be located with the fetal movement monitoring device 1402.

The communication module 1430 may control communications within the housing and send the patient data collected from the sensors 1410 to the fetal movement detection controller 1404 via a wired communications bus. Alternatively, when the fetal movement detection controller 1404 is located separately from the housing, the communication module 1430 may communicate with the fetal movement detection controller 1404 using wireless or wired communications. The fetal movement detection controller 1404 may be executed on and/or embodied in a standalone computing device such as a laptop, tablet or smartphone.

The fetal movement detection controller 1404 is configured to apply a fetal movement monitoring process to the patient data to determine if fetal movement occurs. The fetal movement detection controller 1404 has a communication module 1450 to communicate with the fetal movement monitoring device 1402 and a data store 1460 to store data received from the fetal movement monitoring device 1402. The fetal movement detection controller 1404 also has a data display 1470, such as an LCD display, to display results from information collected from the fetal movement monitoring device 1402.

Disclosed is a method of determining fetal movement during pregnancy. The method receives patient data from measurements from a first sensor and a second sensor attached to a patient. Each of the first and second sensors are selected from the set of sensor types consisting of an EMG (Electromyography) sensor, a flex sensor and an accelerometer. The patient data from the second sensor allows detection of fetal movement when physiological signals impede detection of the fetal movement in the patient data from the first sensor. Typically the first sensor is a different sensor type from the second sensor. Determining that fetal movement has occurred is done by processing the received patient data from the first and second sensors in order to display an indication of the determined fetal movement.

Also disclosed is a method of determining fetal movement during pregnancy where determining a type of fetal movement that occurred is done by processing the received patient data from the first and second sensors in order to display the type of fetal movement. Patient data is received from measurements from a first sensor and a second sensor attached to a patient. Each of the first and second sensors being selected from the set of sensor types consisting of an EMG (Electromyography) sensor, a flex sensor and an accelerometer. The patient data from the first sensor allows determination of a type of fetal movement in combination with the patient data from the second sensor.

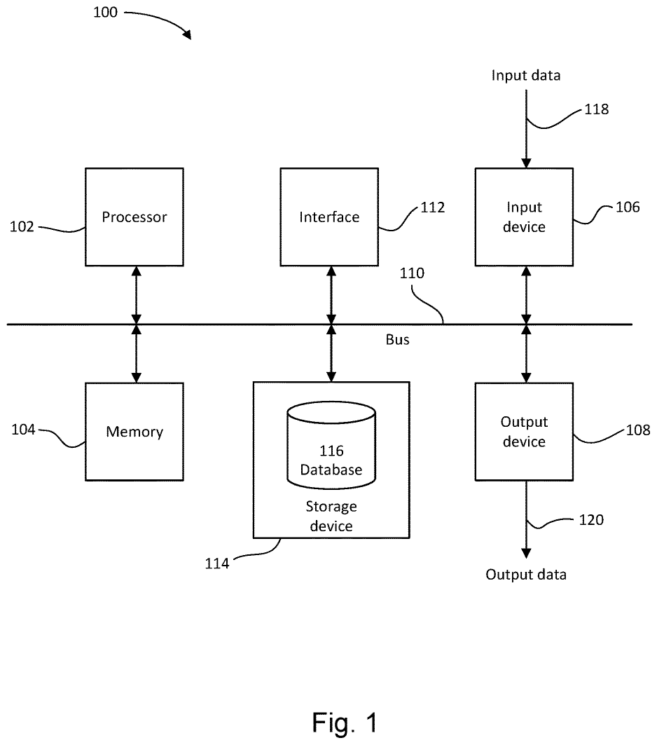

A particular embodiment of the fetal movement monitoring system, or at least one or more components thereof, can be realised using a processing system, an example of which is shown in FIG. 1. In particular, the processing system 100 generally includes at least one processor 102, or processing unit or plurality of processors, memory 104, at least one input device 106 and at least one output device 108, coupled together via a bus or group of buses 110. In certain embodiments, input device 106 and output device 108 could be the same device. An interface 112 can also be provided for coupling the processing system 100 to one or more peripheral devices, for example interface 112 could be a PCI card or PC card. At least one storage device 114 which houses at least one database 116 can also be provided. The memory 104 can be any form of memory device, for example, volatile or non-volatile memory, solid state storage devices, magnetic devices, etc. The processor 102 could include more than one distinct processing device, for example to handle different functions within the processing system 100.

Input device 106 receives input data 118 and can include, for example, a keyboard, a pointer device such as a pen-like device or a mouse, audio receiving device for voice controlled activation such as a microphone, data receiver or antenna such as a modem or wireless data adaptor, data acquisition card, etc. Input data 118 could come from different sources, for example keyboard instructions in conjunction with data received via a network. Output device 108 produces or generates output data 120 and can include, for example, a display device or monitor in which case output data 120 is visual, a printer in which case output data 120 is printed, a port for example a USB port, a peripheral component adaptor, a data transmitter or antenna such as a modem or wireless network adaptor, etc. Output data 120 could be distinct and derived from different output devices, for example a visual display on a monitor in conjunction with data transmitted to a network. A user could view data output, or an interpretation of the data output, on, for example, a monitor or using a printer. The storage device 114 can be any form of data or information storage means, for example, volatile or non-volatile memory, solid state storage devices, magnetic devices, etc.

In use, the processing system 100 is adapted to allow data or information to be stored in and/or retrieved from, via wired or wireless communication means, the at least one database 116. The interface 112 may allow wired and/or wireless communication between the processing unit 102 and peripheral components that may serve a specialised purpose. The processor 102 receives instructions as input data 118 via input device 106 and can display processed results or other output to a user by utilising output device 108. More than one input device 106 and/or output device 108 can be provided. It should be appreciated that the processing system 100 may be any form of terminal, server, specialised hardware, or the like.

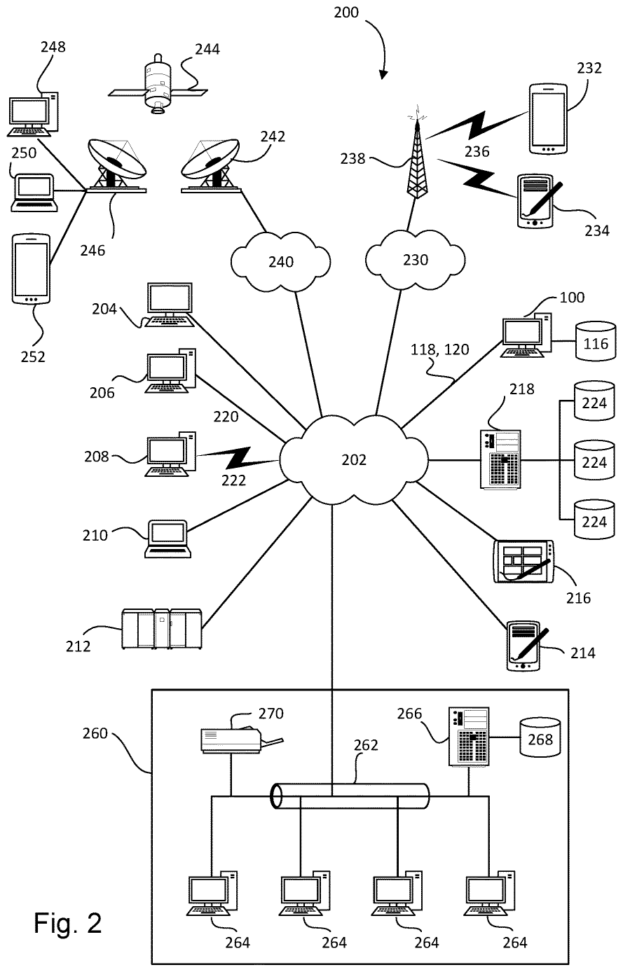

The processing system 100 may be a part of a networked communications system 200, as shown in FIG. 2. Processing system 100 could connect to network 202, for example the Internet or a WAN. Input data 118 and output data 120 could be communicated to other devices via network 202. Other terminals, for example, thin client 204, further processing systems 206 and 208, notebook computer 210, mainframe computer 212, PDA 214, pen-based computer or tablet 216, server 218, etc., can be connected to network 202. A large variety of other types of terminals or configurations could be utilised. The transfer of information and/or data over network 202 can be achieved using wired communications means 220 or wireless communications means 222. Server 218 can facilitate the transfer of data between network 202 and one or more databases 224. Server 218 and one or more databases 224 provide an example of an information source.

Other networks may communicate with network 202. For example, telecommunications network 230 could facilitate the transfer of data between network 202 and mobile, cellular telephone or smartphone 232 or a PDA-type device 234, by utilising wireless communication means 236 and receiving/transmitting station 238. Satellite communications network 240 could communicate with satellite signal receiver 242 which receives data signals from satellite 244 which in turn is in remote communication with satellite signal transmitter 246. Terminals, for example further processing system 248, notebook computer 250 or satellite telephone 252, can thereby communicate with network 202. A local network 260, which for example may be a private network, LAN, etc., may also be connected to network 202. For example, network 202 could be connected with Ethernet 262 which connects terminals 264, server 266 which controls the transfer of data to and/or from database 268, and printer 270. Various other types of networks could be utilised.

The processing system 100 is adapted to communicate with other terminals, for example further processing systems 206, 208, by sending and receiving data, 118, 120, to and from the network 202, thereby facilitating possible communication with other components of the networked communications system 200.

Thus, for example, the networks 202, 230, 240 may form part of, or be connected to, the Internet, in which case, the terminals 206, 212, 218, for example, may be web servers, Internet terminals or the like. The networks 202, 230, 240, 260 may be or form part of other communication networks, such as LAN, WAN, Ethernet, token ring, FDDI ring, star, etc., networks, or mobile telephone networks, such as GSM, CDMA, 4G, 5G etc., networks, and may be wholly or partially wired, including for example optical fibre, or wireless networks, depending on a particular implementation.

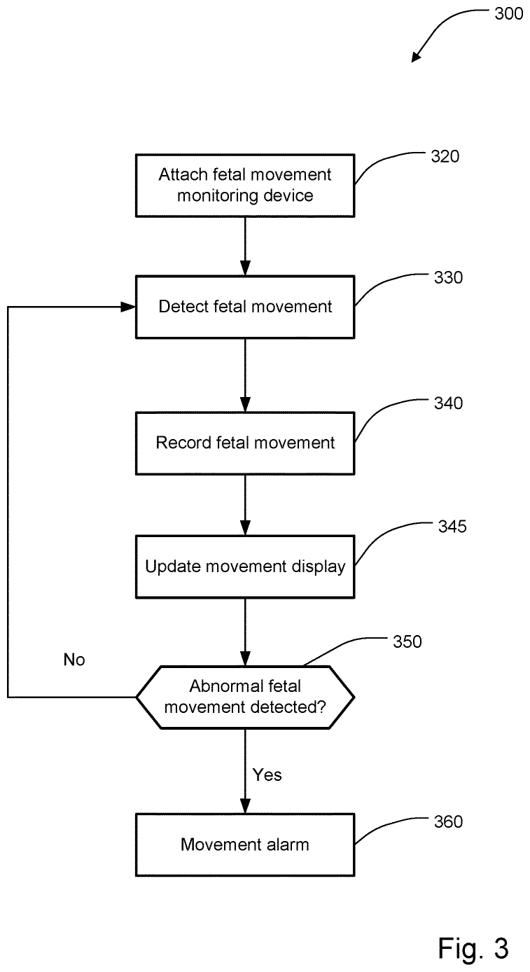

FIG. 3 shows a fetal movement monitoring workflow 300 in which fetal movement monitoring can be performed. The fetal movement monitoring workflow 300 starts when a patient requires, or desires, fetal movement monitoring, such as when presenting to a medical facility or in a home environment. In an apply fetal movement monitoring device subprocess 320, a fetal movement monitoring device is attached to the pregnant individual, typically on the abdomen. This may be done by a clinician, when at a medical facility, or by the patient when in a home environment. As will be described in more detail below, the fetal movement monitoring device may have one or more sensors that can monitor for fetal movement. The fetal movement monitoring device collects output from the one or more sensors and may process the sensor output to detect fetal movement 330. Fetal movement may be recorded in different ways, for example as a binary indication of movement, e.g., movement/no movement, or as a scale of movement where zero indicates no movement and a high value (e.g., ten) indicates large movement of the fetus. In a record fetal movement subprocess 340, the detected fetal movement is stored. Typically the movement value is stored with a time value, such as an absolute or relative time value, to allow information to be determined such as longest pause between fetal movement, shortest pause between fetal movement, average time between fetal movement, etc.

The recorded fetal movement from the record fetal movement 340 may be displayed to the patient, clinician, or other users, on a display device at an update display 345. The display may be a device such as a tablet, smartphone or computer that communicates directly with the fetal movement monitoring device or to another device that communicates with the fetal movement monitoring device. The display may operate as part of the fetal movement detection controller 1404 as described above.

The fetal movement record may be used as part of a check for abnormal fetal movement 350. Typically, there will be predetermined parameters for normal fetal movement. If the recorded fetal movement differs to the predetermined parameters, then the fetal movement may be determined to be abnormal. Alternatively, abnormal fetal movement may be defined by a set of predetermined parameters and the fetal movement is determined to be abnormal if the recorded fetal movement matches the predetermined parameters.

If the check for abnormal fetal movement subprocess 350 determines that the fetal movement is normal, then the fetal movement monitoring workflow 300 returns to the detect fetal movement subprocess 330, and continues to monitor for fetal movement. However, if the check for abnormal fetal movement subprocess 350 determines that there is abnormal fetal movement, the fetal movement monitoring workflow 300 moves to an alarm subprocess 360 where an indication of an audible and/or visual alarm is triggered to alert the user of the fetal movement monitoring device that abnormal fetal movement was detected. In a hospital setting, a clinician can review the fetal movement record and take appropriate action, such as ordering further testing or scans, while in a home setting a user or patient may call for medical assistance. Although not shown, the fetal movement monitoring workflow 300 may continue to detect fetal movement in the detect fetal movement subprocess 330 after the alarm subprocess 360 is initiated and the alarm is raised.

In some implementations, the check for abnormal fetal movement subprocess 350 and the alarm subprocess 360 are optional subprocesses.

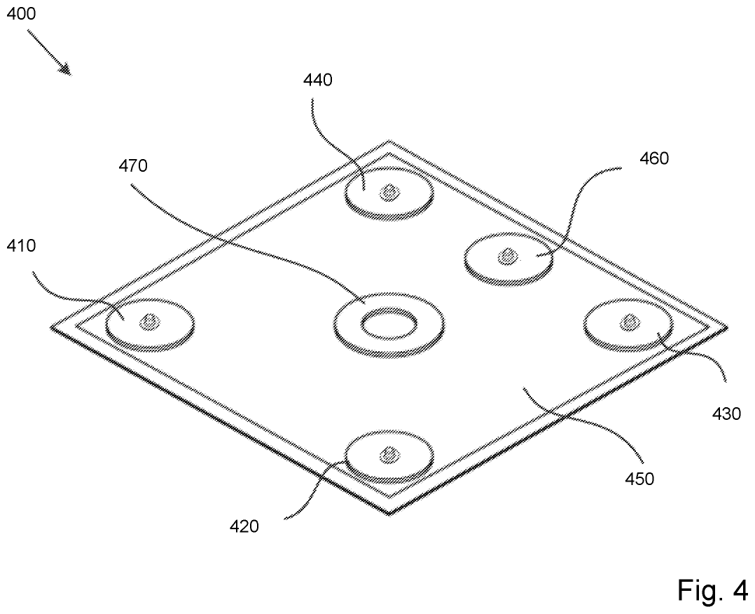

Referring to FIG. 4, there is provided an example of an electrode assembly 400 (which may also be referred to as an “electrode sheet”), which includes a plurality of medical electrode members 410, 420, 430 and 440; and at least one covering sheet 450 removably attached to the plurality of medical electrode members 410, 420, 430 440 and 460. Each of the medical electrode members 410, 420, 430, 440 and 460 may have the same structure as a known medical electrode device.

In this example, the medical electrode member 410 includes a flexible sheet (also referred to as an “electrode backing”) adaptable to the contour of the skin of a patient. The flexible sheet is made of an insulating material, e.g., cloth, plastic, closed cell foam, or any other suitable insulating material that does not conduct electrically, e.g., the electrode backing material can be a foam-and-plastic combination including an adhesive flexible scal that is adhered on top of the flexible sheet around an electrode connector.

A non-electrode adhesive pad 470 is located at or proximate to the centre of the covering sheet 450. Alternatively, the non-electrode adhesive pad 470 may be located in any other location on the covering sheet 450 that allows supporting the medical device. Further, the non-electrode adhesive pad 470 may have any other shape, as long as it allows the non-electrode adhesive pad 470 to adhere to the patient's body and support the medical device on attachment.

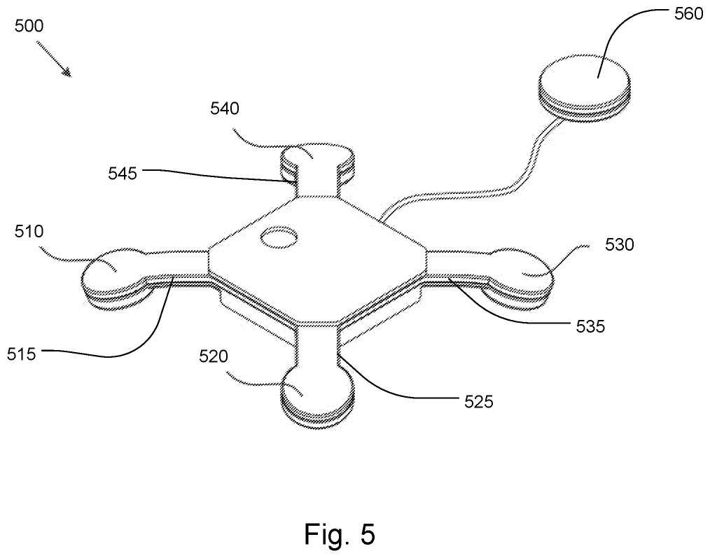

Referring now to FIG. 5 where an example of a fetal movement monitoring device is provided by way of a medical device 500 that can be used together with the electrode assembly 400 shown in FIG. 4, an example of which is the OLI(TM) device to which a plurality of electrode members 410 can be fastened and is described in Australian Provisional Patent Application No. 2016905046 titled “Apparatus for monitoring pregnancy or labour” and/or in PCT Application No. PCT/AU2017/051346 of the same name. The entire contents of the PCT Application No. PCT/AU2017/051346 is incorporated herein by reference. The assembly of the medical device 500 and the electrode assembly 400 creates a fetal movement monitoring device that attaches to a patient. The medical device 500 typically has a power source, such as a battery, to power internal components. The power source may also be used to power a communications system in the medical device 500 that may communicate wirelessly or over wires with a fetal movement detection controller. The medical device 500 attaches individually to each of the electrodes of the electrode assembly 400, allowing each electrode to be electrically connected individually to the medical device 500. The individual electrical, and mechanical, connection allows a reading from each electrode to be taken in isolation from readings from the other electrodes.

The medical device 500 includes a plurality of electrode connecting portions 510, 520, 530, 540 and 560, with the electrode connecting portions 510, 520, 530 and 540 being located at an end of flexible arm portions 515, 525, 535 and 545. One or more of the flexible arm portions 515, 525, 535 and 545 may include a flex or stretch sensor to monitor movement of the electrode connecting portions 515, 525, 535 and 545. Each of the electrode connecting portions is adapted to be connected to a corresponding one of the medical electrode members 410, 420, 430, 440 and 460. Accordingly, the relative positions of the medical electrode members 410, 420, 430, 440 and 460 in the electrode assembly 400 may be arranged based on the relative positions of the corresponding electrode connecting portions 510, 520, 530, 540 and 560 on the medical device 500. As such, it would be understood by the skilled addressee that variations to the shape and arrangement of the features of the of the corresponding electrode connecting portions 510, 520, 530, 540 and 560 in accounting for relative positions of the medical electrode members 410, 420, 430, 440 is within the scope of the invention as described and defined in the claims.

The electrode members 410, 420, 430, 440 and 460 are mutually spaced apart in the electrode assembly 400 to mitigate mechanical and electrical interference between adjacent ones of the electrode members 410, 420, 430, 440. The electrode members 410, 420, 430, 440 are also spaced apart to connect to selected points on the skin, depending on the particular medical procedure and medical device 500. Example dimensions of the electrode assembly 400 can be about 100 millimetres (mm) between electrode members along each side, i.e., a square with sides over 100 mm. Example spacing of the electrode members 410, 420, 430, 440 can be over 50 mm centre-to-centre, e.g., 100 mm between centres, e.g., about 100 mm between centres along each side of the square arrangement in FIG. 4.

The term “patient” used in this disclosure includes both human and animal patients and users. Accordingly, the medical device 500 may include medical devices, well-being equipment and sport-monitoring equipment, for humans or veterinary devices for animals.

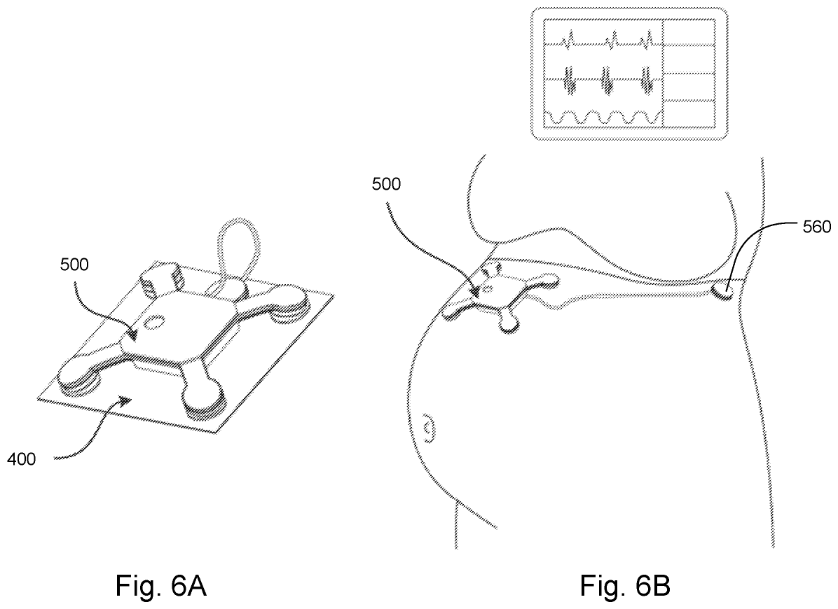

A user can attach the electrode assembly 400 to the medical device 500, as shown in FIG. 6A, to form a fetal movement monitoring device. This may occur by pressing the electrode assembly 400 and the medical device 500 together to mechanically and electrically connect the electrodes to the medical device 500. The electrode assembly 400 may be connected to the medical device 500 so that each of the medical electrode members is secured to a corresponding one of the electrode connecting portions. The user may fasten the fastener (made of a conductive material (e.g., metal), and of a fastener type including a snap fastener (male or female), a tab, a wire or a custom connector) of an electrode connector of each medical electrode member (410, 420, 430, 440 or 460) to a cooperating fastener (which is made of a conductive material (e.g., metal), and of a cooperating type to the fastener's type, e.g., a snap fastener (female or male), a tab, a wire or a custom connector respectively) on a corresponding electrode connecting portion of the medical device 500.

Alternatively, or additionally, the user may adhere the flexible sheet of each medical electrode member (410, 420, 430, 440, or 460) to a flat surface on the corresponding electrode connecting portion of the medical device 500. In addition, the user may further fasten or adhere the non-electrode adhesive pad 470 to a corresponding electrode connecting portion of the medical device 500. In a further embodiment, the method may further include detaching a perforated section of the second covering sheet that is connected to at least one of the plurality of medical electrode members and attaching the at least one of the electrode assembly to a patient's body or a medical device.

The user peels off or removes the second covering sheet from the electrode assembly 100, to expose a patient-side adhesive layer of each medical electrode member (410, 420, 430, 440 or 460), and the patient-side adhesive layer of the non-electrode adhesive pad 470. The user then attaches the medical device 500 with the plurality of medical electrode members to a patient's body such that the plurality of medical electrode members are secured to the patient's body. For example, the medical device 500 with the medical electrode members 410, 420, 430, 440, 460 and the patient-side adhesive layer of the non-electrode adhesive pad 470 is applied to the patient's body as shown in FIG. 6B, e.g., to the abdomen. The medical device 500, along with the medical electrode members 410, 420, 430, 440, 460 and the non-electrode adhesive pad 470 are secured to the patient's body through the adhesive layer of each medical electrode member and the adhesive layer of the non-electrode adhesive pad 470. That is, each of the electrodes are attached to the abdomen of the patient and can take readings from the patient.

After being secured to the patient's body, the medical device 500 and the medical electrode members 410, 420, 430, 440 and 460 can be used to monitor or stimulate the patient, as shown in FIG. 6B. The medical device 500 monitors the electrical signals captured by the medical electrode members 410, 420, 430, 440 and 460, or outputs electrical signals to the medical electrode members 410, 420, 430, 440 and 460 for stimulating the patient's body. The fetal movement monitoring device may collect patient data using an electrical potential sensor, such as an EMG (Electromyography) sensor and/or other sensors, such as a patient response sensor implemented as a temperature sensor operating through the electrode member 410, 420, 430, 440 and 460. Other sensors may be built in to the medical device 500 such as a movement sensor implemented as an accelerometer. Additional patient response sensors may be built into the fetal movement monitoring device. After use, the medical device 500 and the medical electrode members 410, 420, 430, 440 or 460 can be removed from the patient's body

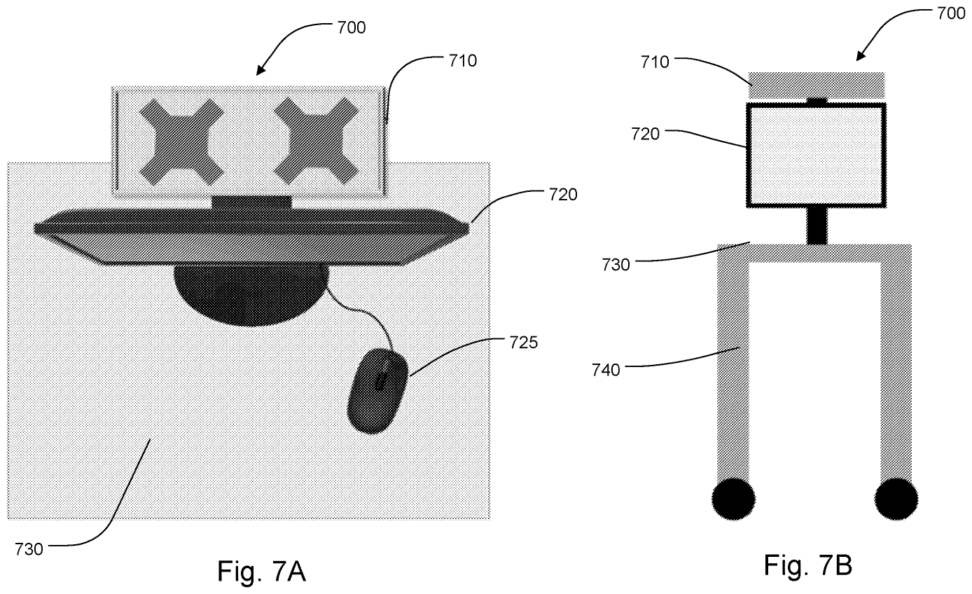

A device station 700 will now be described with reference to FIGS. 7A and 7B. The device station 700 provides a charging location and storage for a fetal movement monitoring device comprising a medical device, such as medical device 500, and an electrode assemble such as the electrode assembly 400. The device station 700 also has a user interface for a user of the fetal movement monitoring system and a preparation area for assembling the fetal movement monitoring device.

The device station 700 has a charge station 710 where an integrated power storage module, e.g., a battery pack, for a medical device component of a fetal movement monitoring device may be charged. The charge station 710 may also include data transfer capabilities to allow one or more medical devices to be configured for wireless communication with the device station 700. In one example, a medical device and the device station 700 may be paired for Bluetooth communication when the medical device is connected to the charge station 710. Alternatively, the medical device may be configured to communicate using other wireless communication protocols such as a Wi-Fi protocol from the IEEE 802.11 family of standards.

A user interface 720 may be driven by a fetal movement detection controller executed on a computer such as the processing system 100 communicating over the network 202. A user of the fetal movement monitoring system can interact with the user interface 720 through a user input device such as a mouse 725. The user interface 720 may display device information for the fetal movement monitoring device, such as the device information including charge status of the medical device, connection and data transmission status of each sensor of the electrode assembly, service history and status, device identification, and other general information about the fetal movement monitoring device and components. The user interface 720 may also display clinical information including a diagnostic outcome of fetal movement, maternal heart rate, current heart rate, contraction information and current contraction information. Historical values of the measure may also be displayed. The charge station 710 and the user interface 720 sit on a cart 740 which may include an assembly area 730 where a fetal movement monitoring device comprising an electrode assembly and a medical device may be assembled for use on a patient.

The fetal movement detection controller driving the user interface 720 may receive and store raw data transmitted from the medical device, and analyse the data using machine learning and signal processing techniques. Results from the analysis may be displayed on the user interface 720.

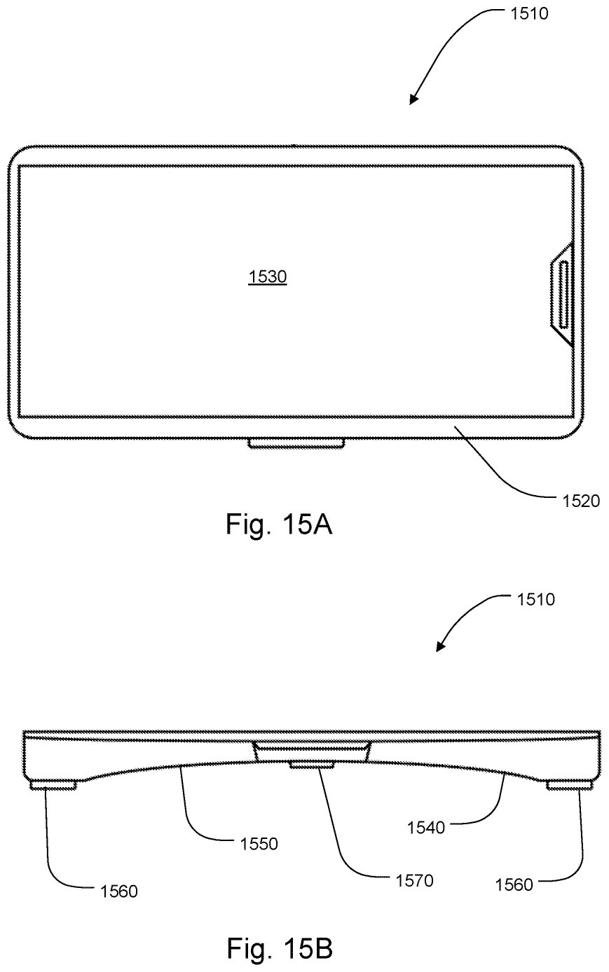

FIGS. 15A and 15B show an alternative medical device 1510 in which fetal movement monitoring can be performed. The medical device 1510 is adapted to be placed on a body and has a housing 1520 which houses electronic components of the medical device 1510. The housing 1520 has a top surface 1530 and a bottom surface 1540. As seen in FIG. 15B, the bottom surface 1540 has a contoured portion 1550 corresponding substantially to a curvature of the maternal abdomen. The housing 1520 is sealed so as to prevent fluid ingress.

The medical device 1510 further comprises a plurality of integrated sensors. The plurality of sensors includes four sensors, three of which are configured to detect different types of signals. In particular, the medical device 1510 includes two electrodes 1560, temperature sensor 1570, and an accelerometer, all integrated into the medical device 1510. In one embodiment one or more flex or stretch sensors may be attached to the electrodes 1560 to determine movement of the electrodes 1560 relative to the housing 1520. The electrodes 1560 are used as part of an EMG sensor.

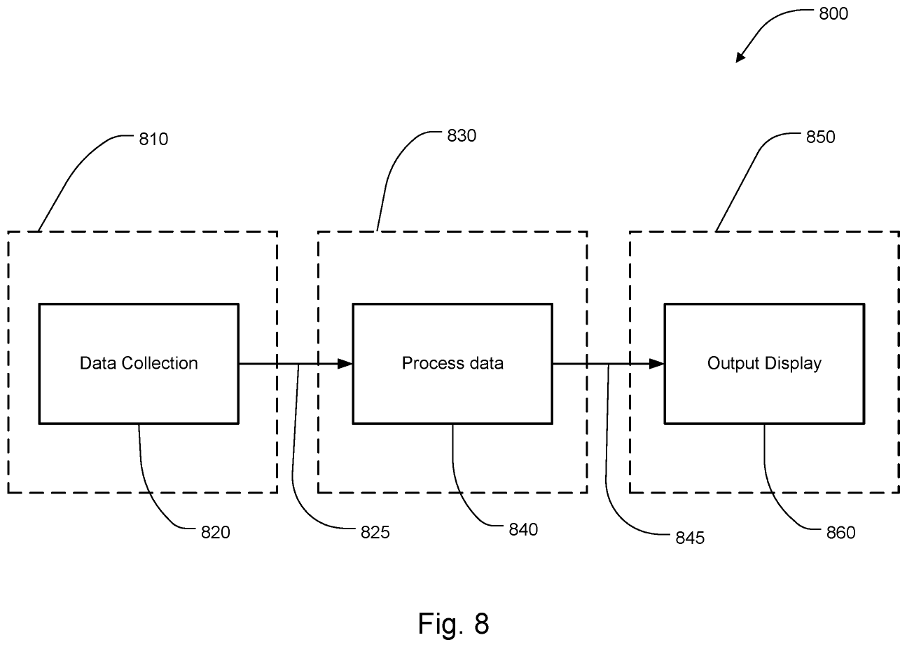

A data processing overview for data from the fetal movement monitoring device will now be described in relation to FIG. 8 which shows a fetal movement data processing method 800 for a patient. The fetal movement monitoring data processing method 800 has three parts. The first part is executed on a fetal movement monitoring device 810, such as the fetal movement monitoring device 1402 described above in FIG. 14, where a data collection process 820 occurs. The data collection process 820 generates patient data from sensors of the fetal movement monitoring device 810. The sensors may be one or more sensors of an electrical potential sensor, a movement sensor, or a deformation sensor that can collect real-time patient data. The electrical potential sensor may be an EMG sensor, the movement sensor may be an accelerometer and the deformation sensor may be a flex sensor. The fetal movement monitoring device 810 has a communication module that transmits patient data 825 to a fetal movement monitoring detection controller 830 similar to the fetal movement detection controller 1404, using a wired or wireless connection. The fetal movement monitoring detection controller 830 and display 850 are located separately to the fetal movement monitoring device 810 which is attached to the patient. Typically, the fetal movement monitoring detection controller 830 and the display 850 are executed by a computer with a monitor for the display 850.

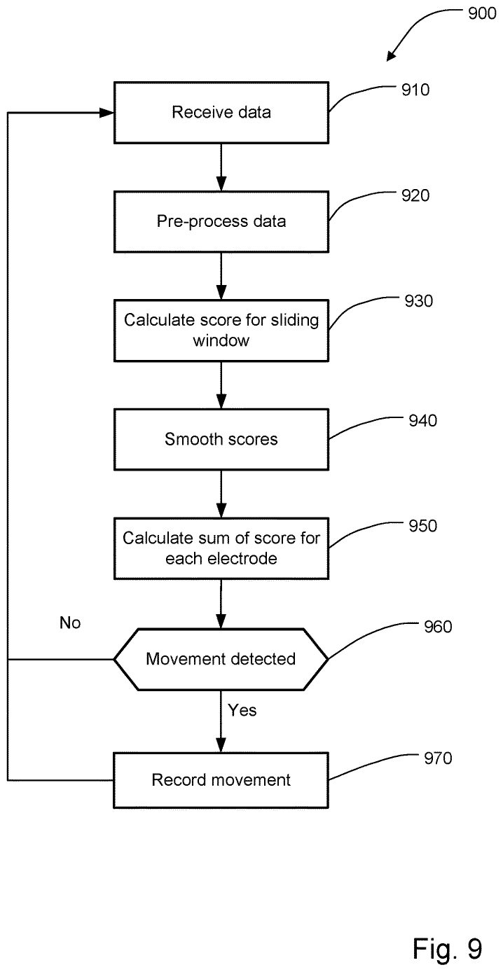

The fetal movement monitoring detection controller 830 receives the patient data 825 as input and analyses the patient data 825 in a process data process 840. Examples of the process data process 840 will be described in more detail in relation to FIG. 9 to FIG. 13. A fetal movement value, or score, determined by the process data process 840 may be compared to a predetermined fetal movement threshold to determine if fetal movement has occurred. When the fetal movement value is less than the threshold, fetal movement has not occurred, and when the fetal movement value is above the threshold, fetal movement has occurred. In one example, the fetal movement value should be above the fetal movement threshold for at least a predetermined time before fetal movement is determined to occur. The output from the fetal movement monitoring detection controller 830 is fetal movement data 845 that may be sent to the display 850 attached to the fetal movement monitoring detection controller 830. The fetal movement data 845 may be displayed on the display 850 as in output process 860. In one example, the output process 860 may provide an indication of a visible display and/or audible alarm on the display 850 when abnormal fetal movement is detected. In another example, the output process 860 may provide information relating the recorded fetal movement on the display 850 such as time since last fetal movement, average time between fetal movement, duration of current record of fetal movement, intensity of fetal movement, etc.

While the display 850 is described above as being attached to the fetal movement monitoring detection controller 830, in one example, the display 850 may be on another device such as a tablet, smartphone, or other computing device, in addition to, or instead of, the fetal movement monitoring detection controller 830. In one example, the fetal movement monitoring detection controller 830 may be integrated into the fetal movement monitoring device 810. The fetal movement data 845 may be transmitted to another device where the fetal movement data may be communicated to a clinician. The fetal movement monitoring device 810 may also be configured to provide the alarm if abnormal fetal movement is detected, via an audible tone from an inbuilt speaker and/or through an integrated display on the fetal movement monitoring device 810. When the fetal movement monitoring device 810 has facilities to provide an alarm, both the fetal movement monitoring detection controller 830 and the display 850 may be integrated into the fetal movement monitoring device 810, e.g., in its housing. In one example, the fetal movement monitoring device 810 may include the fetal movement monitoring detection controller 830 and the display 850 with the fetal movement data 845 also transmitted to one or more additional devices, such as a smartphone, tablet or other computing device.

An EMG fetal movement monitoring process 900, also referred to as a “fetal movement monitoring process”, will now be described in relation to FIG. 9. The EMG fetal movement monitoring process 900 uses patient data measured by only one sensor type, an EMG sensor with two or more electrodes, and produces a pseudo-probability estimation of fetal movement. The EMG fetal movement monitoring process 900 may be practiced on a fetal movement detection controller executed by a computer such as the processing system 100 communicating over a network 202. The EMG fetal movement monitoring process 900 takes/receives sensor input from a fetal movement monitoring device and provides fetal movement information to a user, such as a clinician. Typically the results from the EMG fetal movement monitoring process 900 are displayed on a monitor communicating with the fetal movement monitoring device.

The EMG fetal movement monitoring process 900 starts in a receive data subprocess 910 and receives EMG data from one or more electrodes. Next, the received EMG data is processed in a pre-process data subprocess 920 where one or more pre-processing subprocesses may be applied to the EMG data. Firstly, the data from the electrodes may be verified to ensure that the EMG data is within an acceptable range for an operational sensor. The EMG data may also have a bandpass filter applied. Examples of suitable bandpass filters include filters allowing the following frequency ranges: 0.1 Hz to 2 Hz or 0.1 Hz to 5 Hz. Noise suppression may also be applied to the EMG data. Suitable noise suppression techniques include raising the EMG data from each electrode to an even power, e.g., 2, 4, 6, or applying the Teager-Kaiser Energy Operator.

The pre-process data subprocess 920 forms an electrode pair value or electrode pair vector, referred to as a vector, for readings taken at a pair of electrodes. Typically this is a difference in values between the two electrodes in the pair of electrodes. In a first example, for a fetal movement monitoring device with four electrodes A, B, C and D a differential process is used to determine a plurality of vectors: AB, AC, AD, BC, BD, and CD. That is, a vector is determined between every electrode pair. In a second example, a limited number of electrode pairs are used, such as AB, BC, CD, and AD. The values of the vectors are processed in the pre-process data subprocess 920. Vectors are determined between electrodes attached to the patient and are for pairs of individual electrodes.

In one alternative, a fetal movement monitoring device has five electrodes A, B, C, D and E. A differential process is used to determine ten vectors AB, AC, AD, AE, BC, BD, BE, CD, CE, and DE. In another alternative, one of the five electrodes may be selected as a reference electrode for use in comparing with respective values from electrode pairs made between electrodes excluding the reference electrode. For example, if electrode E is the reference electrode, then a differential process is used to determine vectors AB, AC, AD, BC, BD, and CD. If electrode B is the reference electrode, the vectors may be determined for AC, AD, AE, CD, CE and DE.

In a calculate score subprocess 930 a score for the EMG data is calculated from the pre-processed EMG data. The score is calculated for a current EMG reading for each electrode from the vector data for two or more vectors. For the first set of example vectors described above, AB, AC. AD, BC, BD, and CD, the EMG reading for electrode A may be determined by combining vectors AB, AC, and AD, electrode B as the combination of AB, BC, and BD, electrode C as the combination of AC, BC, and CD, and electrode D as the combination of AD, BD, and CD. For the second example with vectors AB, BC, CD, and AD, the EMG reading for electrode A is the combination of AB and AD, electrode B is the combination of AB and BC, electrode C is the sum combination of BC and CD, and electrode D is the combination of CD and AD. The vectors may be combined as a sum or using other functions to combine the vectors, e.g., linear mathematical relationships, such as subtraction or multiplication. The vectors are combined for vector pairs having a common electrode.

For the five electrode alternative, the score for the EMG reading can be determined from the vector data. When ten vectors are determined, the score can be determined for each of the five vectors, as described above, such as by combining vectors AB, BC, BD, and BE for the EMG reading for electrode B. When one electrode is selected as a reference electrode the score may be determined by combining the vectors that exclude the reference electrode. For electrode E, the vectors AB, AC, AD, BC, BD, and CD may be combined for the score for electrodes A, B, C or D. The score for the electrodes A, B, C or D, generated from the electrode pairs, may be compared to the reference electrode readings, with the reference electrode being used to reduce, or filter out, background signals from the patient.

A sliding window is applied to the EMG data for each electrode, with the 1, 2, 3, 4 or 5 second sliding window being located such that all readings in the window are earlier than a most recent reading. Alternatively, the window may be located about the EMG data reading. A score is produced using the pre-processed EMG data in the window. An example of such a score is a z-score where the current EMG data reading is compared to an average of the EMG data values in the window. In a smooth scores subprocess 940 the scores produced by the calculate score subprocess 930 are smoothed over time. The smoothing may be determined using a technique such as a sliding window mean, also known as a moving average, with a selected window size, e.g., of 1, 2, 3, 4 or 5 seconds. The mean of the scores within the window may be used as the smoothed score. Alternatively a convolution based smoothing filter, such as a Gaussian filter, may be applied to smooth the score.

In a calculate sum of scores subprocess 950 a sum of the smoothed scored for each electrode is determined for a score sum time period. The score sum time period may be the same or different to the window size used for the smooth scores subprocess 940. The fixed time period may be 1, 2, 3, 4 or 5 seconds.

At a check for movement subprocess 960, a predetermined movement threshold is applied to the sum of the scores from the calculate sum of scores subprocess 950. The movement threshold is learnt from, or selected based on, previous patient data, either for the current patient or for other patients. The summed score for each electrode is compared to the movement threshold to determine if the score for each electrode exceeds the movement threshold. If an electrode exceeds the movement threshold for a duration of time, referred to as a trigger window, then fetal movement is determined for the electrode. Determining movement for the electrode allows a fetal movement location to be determined based on the location of the electrode. The trigger window may be ½, 1, or 2 seconds and may be determined using previous patient data. The value of the trigger window and the movement threshold are typically determined together as the two values interact. A higher movement threshold is typically required when a shorter trigger window is selected.

When there is more than one electrode the results may be combined in a suitable manner, for example fetal movement may be detected if fetal movement is determined by a single electrode. Alternatively, fetal movement may be detected if a majority of electrodes determine there is fetal movement. Alternatively, each electrode may have a predetermined weighting applied with electrodes in certain locations on the patient having a higher weighting than other electrodes. The result may be that a fetal movement monitoring device with three electrodes will detect fetal movement if a single electrode with a high weighting determines there is fetal movement, but requires fetal movement detected by two electrodes with a lower weighting to determine fetal movement. The number of electrodes required to determine fetal movement detection may be based on the number of electrodes operating in the fetal movement monitoring device.

If no fetal movement is determined at the check for movement subprocess 960, the EMG fetal movement monitoring process 900 returns to the receive data subprocess 910. If movement is determined in the check for movement subprocess 960 the EMG fetal movement monitoring process 900 proceeds to a record movement subprocess 970 where movements are recorded along with time information. After the record movement subprocess 970 the process EMG fetal movement monitoring process 900 returns to the receive data subprocess 910 to continue monitoring for fetal movement. The EMG fetal movement monitoring process 900 takes patient data as input to determine the fetal movement has occurred. An indication of fetal movement may be displayed to a user as described below.

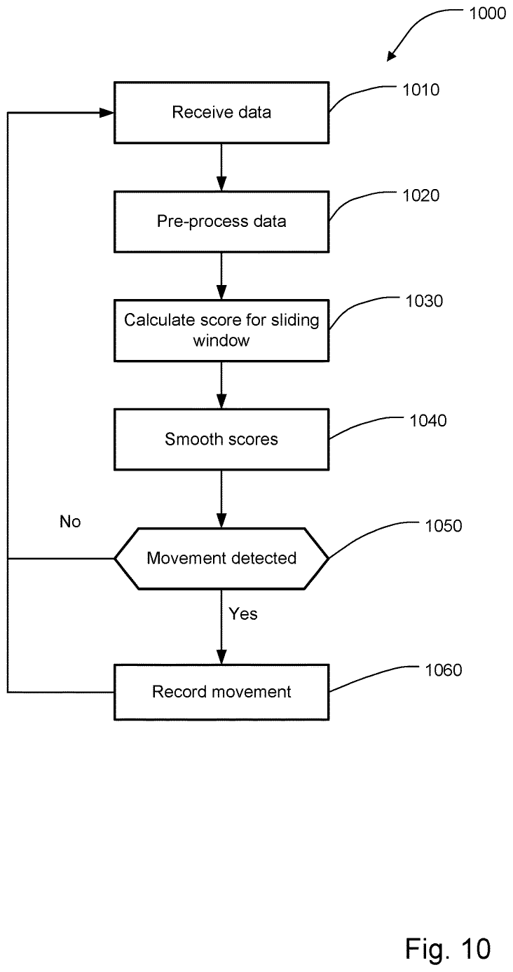

A flex sensor fetal movement monitoring process 1000, which is a fetal movement monitoring process, will now be described in relation to FIG. 10. The flex sensor fetal movement monitoring process 1000 uses patient data measured by only one sensor type, a flex sensor, to detect fetal movement. The flex sensor fetal movement monitoring process 1000 may be practiced on a fetal movement detection controller executed by a computer such as the processing system 100 communicating over a network 202. The flex sensor fetal movement monitoring process 1000 takes/receives sensor input from a fetal movement monitoring device and provides fetal movement information to a user, such as a clinician. Typically the results from the flex sensor fetal movement monitoring process 1000 are displayed on a monitor communicating with the fetal movement monitoring device.

The flex sensor fetal movement monitoring process 1000 starts with a receive data subprocess 1010 and receives flex data from one or more flex sensors. Next, the received flex data is processed in a pre-process data subprocess 1020 where one or more pre-processing steps may be applied to the flex data. Firstly, the data from each flex sensor may be verified to ensure that the flex data is within an acceptable range for an operational sensor. The flex data may also have a high pass filter applied. Examples of suitable high pass filters includes: 0.3 Hz, 0.4 Hz or 0.5 Hz. As with the EMG data, noise suppression may also be applied to the flex data. Suitable noise suppression techniques include raising the flex data to an even power, e.g., 2, 4, 6, or applying the Teager-Kaiser Energy Operator to the flex data.

The flex data is then processed by a calculate score subprocess 1030, a smooth scores subprocess 1040, a check for movement subprocess 1050 and a record movement subprocess 1060. Each of these subprocesses operate in a manner similar to the corresponding subprocesses described in relation to the EMG fetal movement monitoring process 900 above with flex data being processed instead of EMG data. As with the electrodes, the fetal movement monitoring device may use one or more flex sensors. The flex sensor fetal movement monitoring process 1000 takes patient data as input to determine the fetal movement has occurred. An indication of fetal movement may be displayed to a user as described below.

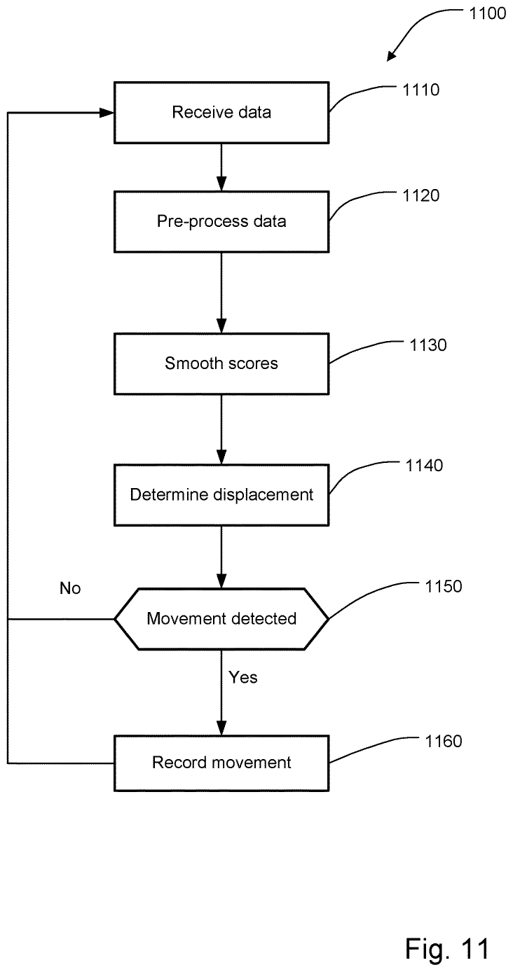

An accelerometer fetal movement monitoring process 1100, which is a fetal movement monitoring process, will now be described in relation to FIG. 11. The accelerometer fetal movement monitoring process 1100 uses patient data measured by only one sensor type, an accelerometer, to detect fetal movement. The accelerometer fetal movement monitoring process 1100 may be practiced on a fetal movement detection controller executed by a computer such as the processing system 100 communicating over a network 202. The accelerometer fetal movement monitoring process 1100 takes/receives sensor input from a fetal movement monitoring device and provides fetal movement information to a user, such as a clinician. Typically the results from the accelerometer fetal movement monitoring process 1100 are displayed on a monitor communicating with the fetal movement monitoring device.

The accelerometer fetal movement monitoring process 1100 starts with a receive data subprocess 1110 and receives accelerometer data from one or more accelerometers. Next, the received accelerometer data is processed in a pre-process data subprocess 1120 where one or more pre-processing subprocesses may be applied to the accelerometer data. Firstly, the data from each accelerometer may be verified to ensure that the accelerometer data is within an acceptable range for an operational accelerometer. The accelerometer data may also have a bandpass filter applied. Examples of suitable bandpass filters includes: 0.5 to 20 Hz, 1 to 10 Hz or 1 to 5 Hz. As with the EMG data, noise suppression may also be applied to the accelerometer data. Suitable noise suppression techniques include raising the accelerometer data to an even power, e.g., 2, 4, 6, or applying the Teager-Kaiser Energy Operator to the accelerometer data.

The pre-processed data from the pre-process data subprocess 1120 is smoothed in a smooth scores subprocess 1130. The data is smoothed over time using a technique such as a sliding window mean, also known as a moving average, with a window size of 2, 3, 4, 5, 6, 7, 8, 9 or 10 seconds. The mean of the scores within the window may be used as the smoothed data.

The data from each accelerometer is a measure of acceleration that is converted to a relative displacement in a determine displacement subprocess 1140. The displacement is determined by integrating the acceleration to determine velocity, then integrating the velocity to determine the displacement. The displacement from the determine displacement subprocess 1140 is used in a check for movement subprocess 1150 where fetal movement may be detected by comparing the relative displacement with a predetermined displacement threshold. As with the check for movement subprocess 960 and the check for movement subprocess 1050, the displacement threshold is learnt, or selected based on, from previous patient data. The relative displacement for each accelerometer is compared to the displacement threshold to determine if the displacement from each accelerometer exceeds the displacement threshold. If the displacement exceeds the displacement threshold for a duration of time, then fetal movement is determined for the accelerometer. The trigger window for fetal movement detected using an accelerometer may be ½, 1, or 2 seconds and may be determined using previous patient data. As with the value of the trigger window and the movement threshold for the EMG and flex sensor, a higher displacement threshold is typically required when a shorter trigger window is selected.

Results from more than one accelerometer may be combined in a suitable manner, as described in relation to the electrodes above for the EMG fetal movement monitoring process 900. If no fetal movement is determined at the check for movement subprocess 1150, then the accelerometer fetal movement monitoring process 1100 returns to the receive data subprocess 1110. If movement is determined at the check for movement subprocess 1150 then the accelerometer fetal movement monitoring process 1100 proceeds to a record movement subprocess 1160 where movements is recorded along with time information. After the record movement subprocess 1160, the accelerometer fetal movement monitoring process 1100 returns to the receive data subprocess 1110 to continue monitoring for fetal movement. The processing system 100 takes patient data as input to determine the fetal movement has occurred. An indication of fetal movement may be displayed to a user as described below.

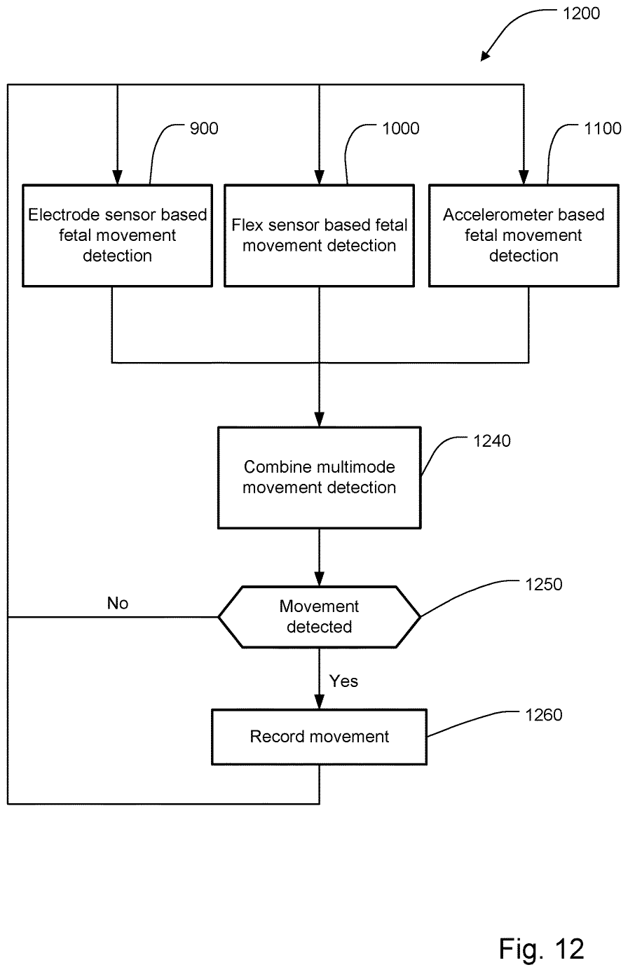

A multi-sensor fetal movement monitoring process 1200 will now be described in relation to FIG. 12. The multi-sensor fetal movement monitoring process 1200 combines patient data from two or more of the previously described fetal movement monitoring processes of the EMG fetal movement monitoring process 900, the flex sensor fetal movement monitoring process 1000 and the accelerometer fetal movement monitoring process 1100. The multi-sensor fetal movement monitoring process 1200 may be practiced on a fetal movement detection controller executed by a computer such as the processing system 100 communicating over a network 202. The multi-sensor fetal movement monitoring process 1200 takes/receives sensor input from a fetal movement monitoring device and provides fetal movement information to a user, such as a clinician. Typically the results from the multi-sensor fetal movement monitoring process 1200 are displayed on a monitor communicating with the fetal movement monitoring device.

The multi-sensor fetal movement monitoring process 1200 shows three possible sources of fetal movement detection from three different sensor types, the EMG fetal movement monitoring process 900, the flex sensor fetal movement monitoring process 1000 and the accelerometer fetal movement monitoring process 1100. While all three fetal movement monitoring processes are shown, two or more processes may be used, in any combination. For example, output from an EMG may be combined with output from an accelerometer using the EMG fetal movement monitoring process 900 and the accelerometer fetal movement monitoring process 1100. In a similar way the output from one or more accelerometers may be combined with output from one or more flex sensors. Output from an EMG and a one or more flex sensors may be combined or output from one or more electrodes, flex sensors and accelerometers may also be combined.

The output from two or more of the fetal movement detection processes are combined in a movement detection combination subprocess 1240. The output of the fetal movement detection processes may be combined using techniques such as boosting and bagging, where the parameters are learnt using, or selected based on, previous patient data. Alternatively, the outputs may have a logical OR applied, so that if movement is detected separately in any one of the fetal movement detection processes, then that fetal movement is deemed to be detected. That is, at least a first sensor and a second sensor are processed separately to determine that fetal movement has occurred and the determination of fetal movement based on at least the first sensor and the second sensor are combined to determine fetal movement. A third sensor may also be combined with the first and the second sensors.

At check for movement 1250, when no fetal movement is detected, the multi-sensor fetal movement monitoring process 1200 loops the fetal movement detection processes to collect further data. However, when fetal movement is determined to occur in the movement detection combination subprocess 1240, the multi-sensor fetal movement monitoring process 1200 moves to a record movement subprocess 1260 where fetal movement information is recorded. Information may include, time of movement as well as which fetal movement monitoring process detected fetal movement. After the record movement subprocess 1260, the multi-sensor fetal movement monitoring process 1200 loops to execute the fetal movement detection processes to collect further fetal movement data.

In an alternative embodiment, the multi-sensor fetal movement monitoring process 1200 may operate in a similar manner as described above. However, the EMG fetal movement monitoring process 900, the flex sensor fetal movement monitoring process 1000 and the accelerometer fetal movement monitoring process 1100 may provide a processed, or unprocessed, form of the patient data as intermediate data, such as pre-processed patient data or smoothed scores. The pre-processed data is the output of the pre-process data subprocess 920, the pre-process data subprocess 1020 or the pre-process data subprocess 1120. The smoothed scores is the output of the smooth scores subprocess 940, the smooth scores subprocess 1040 or the smooth scores subprocess 1130. Alternatively, the intermediate data may be a summed score for an electrode from the calculate sum of scores subprocess 950 or the displacement from the determine displacement subprocess 1140. In one embodiment the unprocessed patient data from the sensors may be output by the EMG fetal movement monitoring process 900, flex sensor fetal movement monitoring process 1000 and the accelerometer fetal movement monitoring process 1100.

The intermediate patient data, unprocessed or processed, may be combined in the movement detection combination subprocess 1240 to determine fetal movement and/or to determine a type or classification of the fetal movement detected. The fetal movement type may be determined by processing the received patient data from two or more sensors. The patient data from the two or more sensors being combined to determine the fetal movement type. Examples of how intermediate data, in the form of processed or unprocessed patient data, are combined are described below. In one example the sensors used may be of a same type, however in another example two or more different sensor types may be used.

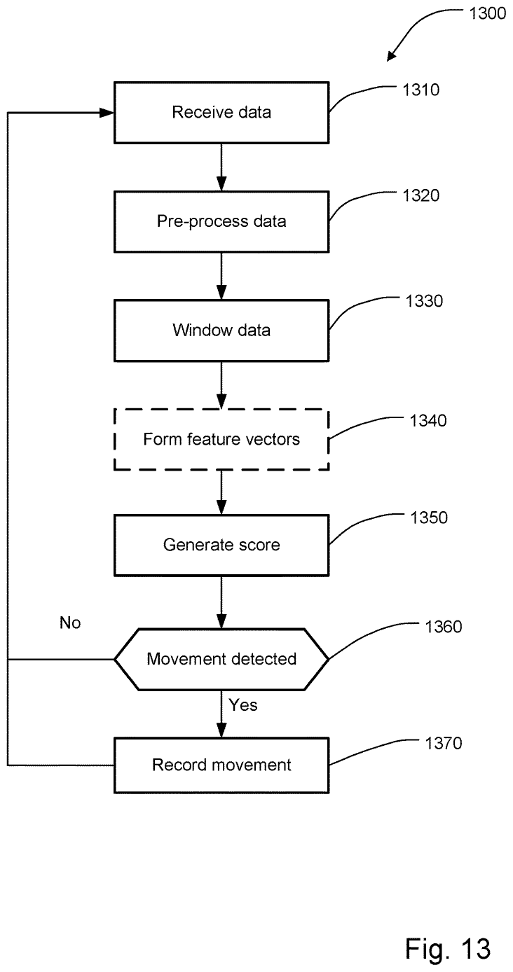

An alternative multi-sensor fetal movement monitoring process 1300, which is a fetal movement monitoring process, will now be described in relation to FIG. 13. The alternative multi-sensor fetal movement monitoring process 1300 uses patient data measured by two or more sensor types, selected from the set of EMG, one or more flex sensors and one or more accelerometers, to detect fetal movement. The alternative multi-sensor fetal movement monitoring process 1300 uses a trained classifier to detect fetal movement using a fetal movement monitoring device attached to the patient. The alternative multi-sensor fetal movement monitoring process 1300 may be practiced on a fetal movement detection controller executed by a computer such as the processing system 100 communicating over a network 202. The alternative multi-sensor fetal movement monitoring process 1300 takes/receives sensor input from a fetal movement monitoring device and provides fetal movement information to a user, such as a clinician. Typically the results from the alternative multi-sensor fetal movement monitoring process 1300 are displayed on a monitor communicating with the fetal movement monitoring device. The alternative multi-sensor fetal movement monitoring process 1300 takes patient data from at least a first sensor and a second sensor as input to a trained classifier to determine that fetal movement has occurred

The alternative multi-sensor fetal movement monitoring process 1300 starts with a receive data subprocess 1310 where patient data from the sensors is collected. Next, in pre-process data subprocess 1320, the received sensor data is pre-processed. The pre-processing may take the form of the pre-process data subprocess 920, the pre-process data subprocess 1020 and the pre-process data subprocess 1120, depending on the originating sensor type for the data. An additional subprocess that may be performed is normalisation of data between the different sensor types. The normalisation may be performed by zeroing the mean and setting a uniform standard deviation or whitening.

In window data subprocess 1330, a window of pre-processed data is selected using a fixed time window that may be 1, 2, 3, 4, 5, 6, 7, 8, 9, or 10 seconds long. Next, the windowed data may be formed into a feature vector at an optional form feature vectors subprocess 1340. The windowed data may be arranged as a high dimensional feature vector to form a feature vector from patient data collected by a sensor, or at least a first and a second sensor. Statistical measures such as minimum, maximum, mean, interquartile ranges and standard deviation may be used when part of the feature vector generated at the form feature vectors subprocess 1340.

In a generate score subprocess 1350 a trained classifier is applied to either feature vectors from the form feature vectors subprocess 1340 or the windowed data from the window data subprocess 1330. If feature vectors are used, the feature vectors are input into the trained classifier, such as a linear or non-linear machine learning model. If the form feature vectors subprocess 1340 is not use, windowed data from the window data subprocess 1330 is input into the trained classifier. In examples, the linear or non-linear machine learning model may include support vector machines, Gaussian Processes, decision trees, graphical models and neural networks, such as recurrent neural networks and their variations, multilayer perceptron's and convolutional neural networks. The classifier is trained using previous patient data to determine parameters for the classifier. Output from the machine learning model is a score or probability representing a likelihood of fetal movement.

In a check for movement subprocess 1360 a predetermined movement threshold is applied to the score or probability generated at the generate score subprocess 1350. The movement threshold is learnt from previous patient data. The score or probability is compared to the movement threshold to determine if movement was detected by the sensors of the fetal movement monitoring device. If the score or probability exceeds the movement threshold for a duration of time of a trigger window, then fetal movement is determined. The trigger window may be ½, 1, or 2 seconds and may be determined using previous patient data. The value of the trigger window and the movement threshold are typically determined together as the two values interact. A higher movement threshold is typically required when a shorter trigger window is selected.

If no fetal movement is determined at the check for movement subprocess 1360 then the alternative multi-sensor fetal movement monitoring process 1300 returns to the receive data subprocess 1310. If movement is determined at the check for movement subprocess 1360 then the alternative multi-sensor fetal movement monitoring process 1300 proceeds to a record movement subprocess 1370 where movement, time information and the score or probability may be recorded. After the record movement subprocess 1370 the alternative multi-sensor fetal movement monitoring process 1300 returns to the receive data subprocess 1310 to continue monitoring for fetal movement.

While the alternative multi-sensor fetal movement monitoring process 1300 is described as having a separate threshold, the generate score subprocess 1350 may use the classifier to output if fetal movement has occurred or not occurred. When the classifier is configured to determine if fetal movement has occurred or not, the threshold is considered to be part of the classifier.

A multi-modal sensor approach to fetal movement detection, such as the multi-sensor fetal movement monitoring process 1200 and the alternative multi-sensor fetal movement monitoring process 1300, may be beneficial in terms of sensor redundancy by combining patient data from two or more different sensor types. The multi-modal sensor approach may also enable a better understanding of signals being detected that allows identification of fetal movement types and intensities. Additional modalities can assist during the presence of other physiological signals from activities such as respiratory, maternal movement, etc. These activities may be detected by a first sensor, but not detected by a second sensor, or vice versa. The first and second sensors are different sensor types and may be one of an EMG sensor, flex or stretch sensor, or an accelerometer, where the EMG sensor reads values from electrodes. As such, the use of two or more sensors may allow fetal movement detection even in the presence of other physiological signals that prevent, or mask, detection of fetal movement by a sensor type. If only one sensor type is used, the signal of interest may be impeded, hindered or even lost, hidden by or within other physiological signals when they occur.

The following are some examples of a combination of patient data from sensors being used to determine a type and/or intensity of fetal movement:
A first sensor is an EMG sensor, that enables identification of a fetal movement, and a second sensor is an accelerometer that enables a force of the movement to be determined. The force of movement allows differentiation between a sudden vs controlled movement (e.g., roll vs kick, wriggle vs roll, wriggle vs kick etc) to provide fetal movement differentiation.
Alternatively, the first sensor is an EMG sensor, that enables identification of a fetal movement, and the second sensor is a flex that enables different movement to be measured, such as a subtle or strong movement (e.g., roll vs kick, wriggle vs roll, wriggle vs kick etc). This allows determination of fetal movement strength. The flex sensor can differentiate fetal movement by an amount of deformation.
Alternatively, the first sensor is an EMG sensor, that enables identification of a fetal movement, and a second sensor is an additional EMG sensor to provide electrode readings in a comparative location to differentiate between a small localised movement and a larger, spread out, movement (e.g., roll vs kick, wriggle vs roll, wriggle vs kick etc). This allows movement localisation to be determined.
Alternatively, the first sensor is a flex sensor, that enables identification of a fetal movement, and the second sensor is an accelerometer that provides acceleration data for the fetal movement. The acceleration data may differentiate between a sudden or controlled fetal movement (e.g., roll vs kick, wriggle vs roll, wriggle vs kick etc) to provide fetal movement differentiation.
Alternatively, the first sensor is a flex sensor, that enables identification of a fetal movement, and the second sensor is an additional flex sensor that provides deformations data in a comparative location to differentiate between a small localised and a larger spread out movement where both flex sensors would detect the movement (e.g., roll vs kick, wriggle vs roll, wriggle vs kick etc). This allows movement localisation to be determined.
Alternatively, the first sensor is an accelerometer, that enables identification of a fetal movement, and the second sensor is a flex sensor that provide deformation data to determine a scale of the fetal movement allowing differentiation between a subtly movement and a strong movement (e.g., roll vs kick, wriggle vs roll, wriggle vs kick etc). This allows determination of fetal movement strength.
Alternatively, the first sensor is an accelerometer, that enables identification of a fetal movement, and a second sensor is an additional accelerometer that provides acceleration data for a comparative location. The two accelerometers allow differentiation between a small localised fetal movement and a larger spread out fetal movement (e.g., roll vs kick, wriggle vs roll, wriggle vs kick etc). This allows movement localisation to be determined.

As described above, the acceleration data from the accelerometer allows differentiation of the intensity of the fetal movement between at least a small and large movement. Further, the intensity of fetal movement can be determined according to a combination of the patient data from a first and a second sensor. Also, fetal movement type can be determined based on a location of the fetal movement. Additionally, a sensor type of a first sensor and a second sensor can be used to determine fetal movement type.

Additional data may also be received from the patient in the form of maternal input of fetal movement perception. The patient may be provided with an interface for providing information about perceived fetal movement. When the patient perceives fetal movement they indicate detection of the movement using the interface. A time and source of the fetal movement detection is recorded and may be cross referenced, or compared, with information from the fetal movement monitoring device. The cross referencing may allow training of the fetal movement detection using the fetal movement monitoring device information, such as confirming fetal movement detected by the fetal movement monitoring device. The detection of fetal movement by the fetal movement monitoring device may be compared with maternal input of fetal movement perception. Alternatively, the maternal input of fetal movement perception may be used to calibrate or adjust one or more thresholds in a fetal movement detection processes, allowing dynamic tuning, or adjustment, of the thresholds so the thresholds are adaptive to the patient. The dynamic tuning, or adjustment, of the threshold allows for dynamic tuning, or adjustment of the fetal movement detection. In one example, a threshold to determine fetal movement detection is adjusted based on the maternal input of fetal movement perception, where the threshold is too high to detect fetal movement when the maternal input indicates fetal movement has occurred. The interface may be in the form of a wired or wireless physical button provided to the patient, a mobile phone application or other input device. In one example, the patient may tap the fetal movement monitoring device using a predetermined tapping pattern, such as three taps, to indicate fetal movement. The taps are detected by an accelerometer in the fetal movement monitoring device and fetal movement information recorded.

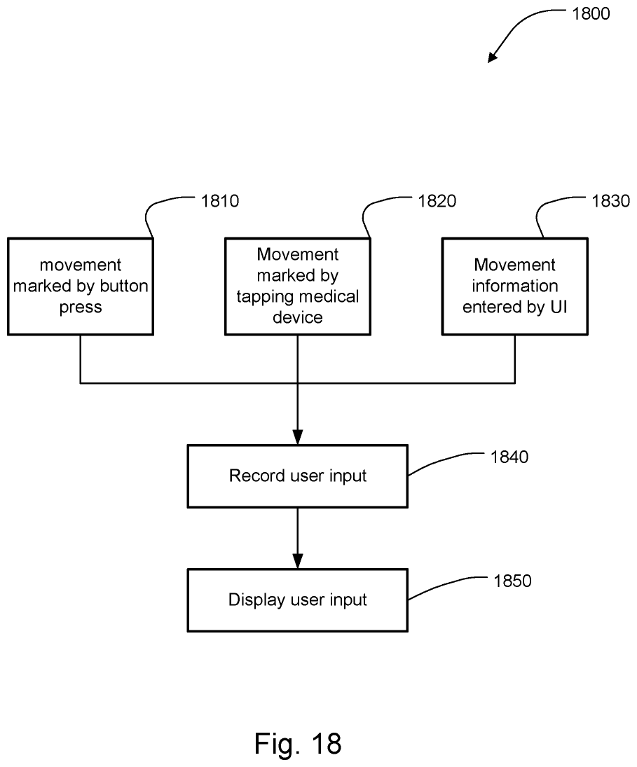

FIG. 18 shows a manual movement marking process 1800 for recording maternal input of movement perception. The manual movement marking process 1800 can take input from three sources, including in a mark movement by button subprocess 1810, a mark movement by tapping subprocess 1820 (including tapping the fetal movement monitoring device) and a mark movement by User Interface subprocess 1830. Maternal input may be recorded by any one of the three inputs. In the store user input subprocess 1840, the user input is recorded and may include information such as a source of the information, time the information was recorded and fetal movement type. In a display user input subprocess 1850, the user input may be displayed. The display user input subprocess 1850 is an optional subprocess, or may occur at a later time than the store user input subprocess 1840.

In one example, the manual movement marking process 1800 may record fetal movement types. The fetal movement types may be recorded by having a button for each movement type for the mark movement by button subprocess 1810, using different tapping patterns for the mark movement by tapping subprocess 1820, or having different buttons on the user interface for the mark movement by UI subprocess 1830.

Maternal presentation information may also be collected and included when determining fetal movement. Maternal presentation information that may impact fetal movement readings and or perception in some way is collected either by input via a user interface, such as the user interface 720 or through integrating with third-party records, such as health record or Epic. The maternal presentation information may be used in determining the fetal movement or used to adjust how fetal movement is determined by the fetal movement monitoring device. In one example of how maternal presentation information is used, a sensitivity for the electrodes is adjusted based on maternal presentation information. In one example, the maternal presentation information can allow thresholds in fetal movement detection processes to be adjust, so the thresholds are set based on the maternal presentation information. That is, the fetal movement detection threshold may be set taking into account maternal presentation information. For example, a fetal movement detection threshold may be lowered to account for a high BMI. In another example, the fetal movement detection threshold may be lowered if the fetal size is small. Each item of the maternal presentation information can provide an adjustment to a threshold, with an overall maternal presentation information threshold adjustment calculated from the sum of the individual adjustments for each item. The maternal presentation information may include items such as Body Mass Index (BMI), placenta location, fetal size, and parity.

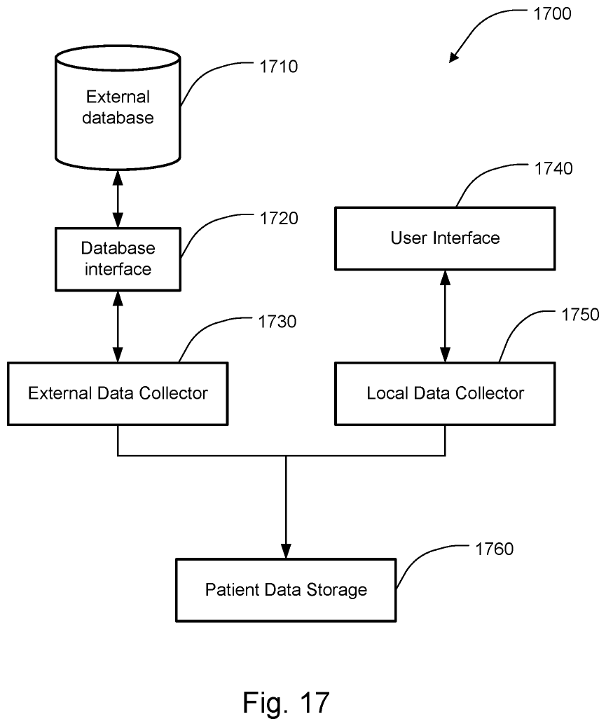

A maternal presentation collection system 1700, as seen in FIG. 17, shows how maternal presentation information may be collected. A first source of information is an external database 1710 that can be accessed using a database interface 1720. Collection of the maternal presentation information from the external database 1710 is controlled by an external data collector 1730. Any information collected by the external data collector 1730 is stored at a patient data storage 1760. A second source of maternal presentation information is collected locally by a clinician or patient entering data at a user interface 1740. A local data collector 1750 takes the information from the user interface 1740 and stores the information at the patient data storage 1760. The external data collector 1730, user interface 1740, local data collector 1750 and the patient data storage 1760 may be practiced on a computer such as the processing system 100 communicating over a network 202.

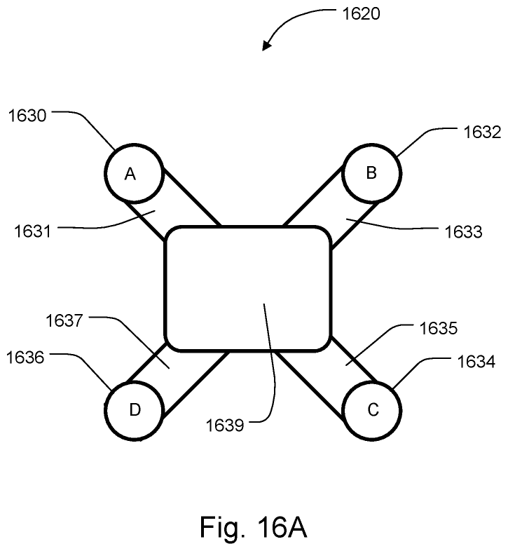

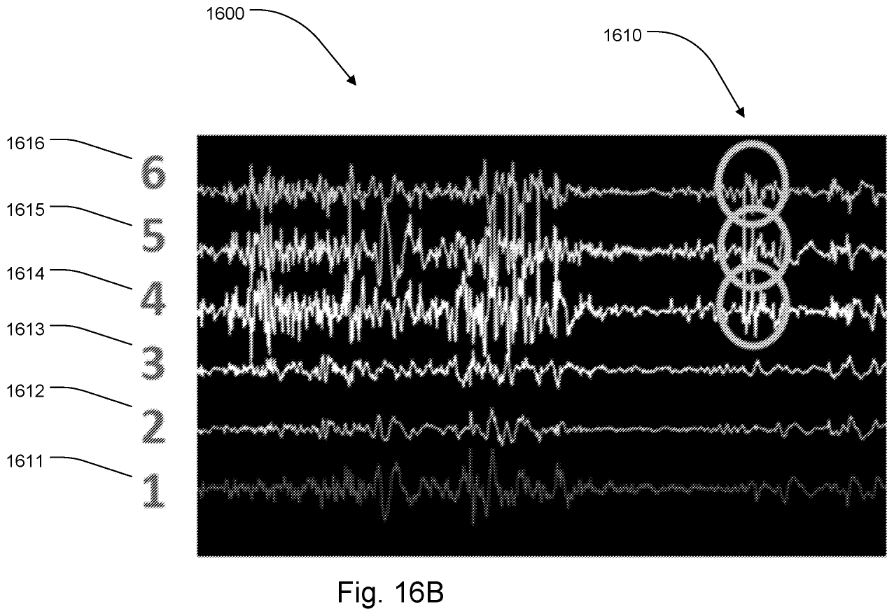

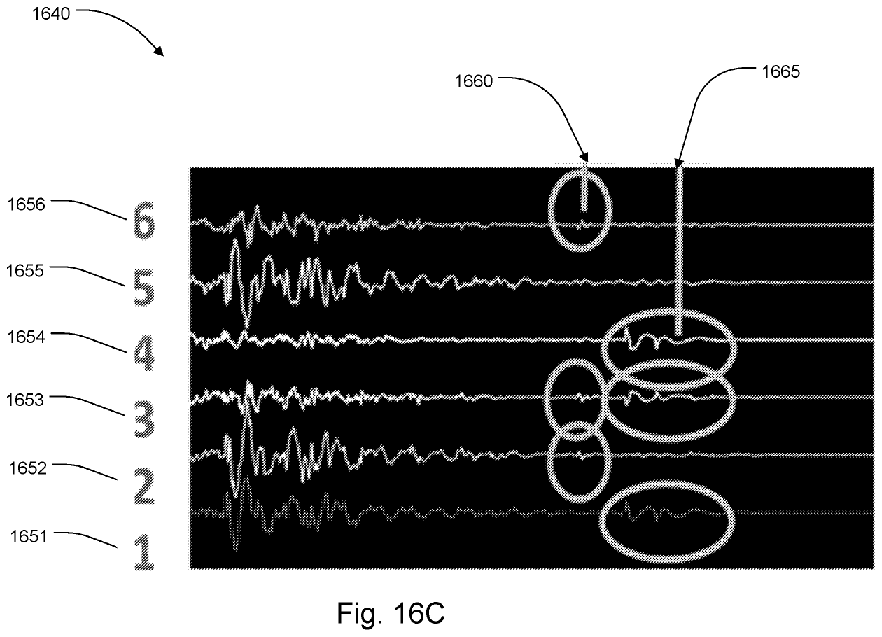

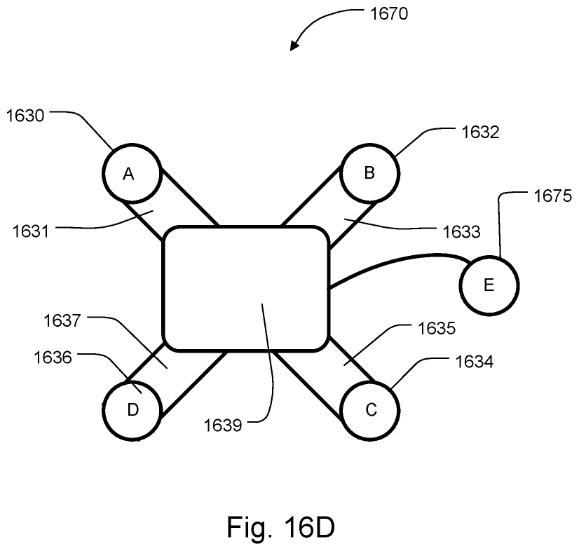

FIGS. 16A, 16B 16C and 16D show how different types of fetal movement may be determined, where the movement is detected at different locations on a patient using patient data. The fetal movement types are determined using a medical device 1620, shown in FIG. 16A, having four electrodes as part of a sensor, electrode A 1630, electrode B 1632, electrode C 1634 and electrode D 1636. Each of the electrodes is located at an end of a flexible arm portion 163, flexible arm portion 1633, flexible arm portion 1635 and flexible arm portion 1637 respectively. The flexible arm portions may each have a flex or stretch sensor to measure bending of the arm portion, while one or more accelerometers may be located in a central portion 1639. As described above, in relation to EMG fetal movement monitoring process 900, the four electrodes are used to generate vectors between each of the electrodes. Six vectors are generated, being AB, AC, AD, BC, BD and DC, where the letters represent the two electrodes such that AB is between electrode A 1630 and electrode B 1632.

Values of the six vectors are shown in FIG. 16B which shows a sensor output chart 1600 that shows the values of the vectors varying over time. The sensor output chart 1600 shows electrode vector AB 1611, electrode vector AD 1612, electrode vector BD 1613, electrode vector BC 1614, electrode vector AC 1615 and electrode vector CD 1616. The values of the six vectors can be compared to determine a location of activity detected by the electrodes of the sensor. Localised activity 1610 shows a time span where similar signal activity is visible on the electrode vector BC 1614, the electrode vector AC 1615 and the electrode vector CD 1616. Electrode C 1634 is common to all three of the vectors, so the activity detected by the electrodes is located at electrode C 1634.

Further combined sensor data output is shown in FIG. 16C that shows an sensor output chart 1640 showing vector values varying over time. The sensor output chart 1640 shows electrode vector AB 1651, electrode vector AD 1652, electrode vector BD 1653, electrode vector BC 1654, electrode vector AC 1655 and electrode vector CD 1656. Also marked are localised activity 1660 which shows similar signal activity occurring on electrode vector AD 1652, electrode vector BD 1653 and electrode vector CD 1656. The activity marked by localised activity 1660 takes place at electrode D 1636 since electrode D 1636 is the common electrode to all three vectors. Similarly, localised activity 1665 shows similar signal activity on electrode vector AB 1651, electrode vector BD 1653 and electrode vector BC 1654, which has electrode B 1632 as the common electrode. Therefore the activity was detected by electrode B 1632.

FIG. 16D shows a medical device 1670 that is similar to the medical device 1620, described above in relation to FIG. 16A. In addition to the electrode A 1630, electrode B 1632, electrode C 1634 and electrode D 1636 the medical device 1670 has electrode E 1675 which provides an additional electrode that may be positioned on the patient. The electrodes 1630, 1632, 1634 and 1636 are located in a relatively fixed arrangement, with small movement possible by the flexible arm portions 1631, 1633, 1635 and 1637. However electrode E 1675 may be positioned anywhere on the patient within reach of lead 1677. The electrode E 1675 may be used as a reference electrode and placed on the body of the patient in a location spaced from the fetal movement monitoring device, where fetal or maternal activity will be absent, so as to provide a reference to the plurality of sensors. An example can be seen for the electrode connecting portion 560 as shown in FIG. 6B. In one alternatively, one of the other electrodes may be selected as the reference electrode.

By comparing the electrode vectors, a source of the signal may be determined. The source of the signals may correspond to a location where movement has occurred, allowing the location of the movement to be determined. In the sensor output chart 1640, the localised activity 1660 and the localised activity 1665 show two different types of fetal movement occurring at two different locations. The difference in fetal movement type may be determined from a difference in the waveforms of the localised activity 1660 and the localised activity 1665 or from a difference in the location of the localised activity 1660 and the localised activity 1665. Such differences may represent a different type of fetal movement, such as a kick, a wiggle, or roll. The differences may also represent a different in intensity of the fetal movement, such as a small impact and low intensity movement versus a high impact and high intensity movement. In one example, a relationship exists between fetal movement type and fetal movement intensity, so that fetal movement type may be determined according to an intensity of the fetal movement. In such an example the fetal movement intensity, such as large movement or small movement, may be used to determine the type of fetal movement, such as a roll or kick.

Determining an electrode where fetal movement is detected may involve determining electrode pair vectors between electrodes of the sensor, such as AB, BC, etc. as described above. The pairs are compared and electrode pair vectors with the same, similar or substantially similar signals selected, as shown in FIGS. 16B and 16C. The location of the fetal movement may be determined by selecting an electrode that is common to the electrode pair vectors selected. The location of the electrode may then be considered to be at, or near, the location of the fetal movement. An additional electrode location may be determined by using the electrode pair vectors to find at least two electrode pair vectors to identify a second electrode to determine a second location where fetal movement is determined to have occurred. As described above, the location of the fetal movement, determined from the sensor configuration, may be used to determine the fetal movement type through determining fetal movement intensity or other means.

Location of the fetal movement may be used as an indication of the fetal movement intensity. For example, detecting fetal movement at multiple locations, in two or more location, can be an indication that the fetal movement was more intense than fetal movement detected from a single electrode. That is, the fetal movement intensity can be determined according to how many electrodes detected the fetal movement. Some electrode locations may require more intense fetal movement to detect fetal movement, compared to other electrodes. As such, the intensity of the fetal movement can also be determined based on a location of the fetal movement. In one example, the fetal movement intensity is determined based a combination of a location of the electrodes that determine the fetal movement as well as how many electrodes detect the fetal movement.

While the sensor output chart 1600 and the sensor output chart 1640 show the output from electrodes of a sensor, similar charts and processing may be produced using a combination of different sensor types. For example, a chart may include output from one or more accelerometers or one or more flex or stretch sensors. Fetal movement type and/or intensity is determined by comparing readings from a combination of different sensor types and using information regarding positions of the accelerometers and flex sensors on a medical device and, ultimately, on the patient. In one example, a flex sensor located in the flexible arm portion 1631 may register bending of the flexible arm portion 1631 at the same time that electrode A 1630 detects fetal movement as described above. Similarly, a flex sensor in the flexible arm portion 1637 may detect bending at the same time as fetal movement is determined for electrode D 1636. In a similar manner, an accelerometer in the central portion 1639 may detect movement for the four electrodes. In one example, the central portion 1639 may house multiple accelerometers, with one accelerometer being located close to each of the electrodes. Such an arrangement may allow the accelerometers to detect acceleration and be used to determine a location of fetal movement close to one of the electrodes. Patient data from the accelerometers, electrodes and the flex sensors may be combined to determine fetal movement in a common region. An example of such a common region is the region near electrode A 1630, that may also use patient date from the flex sensor of the flexible arm portion 1631 as well as accelerometer data for an accelerometer associated with the region. Each of the electrodes, flex sensor and the accelerometer are associated with a common region.

### Display of Fetal Movement

The results of the fetal movement monitoring processes may be displayed on a monitor communicating with the fetal movement monitoring device. One way to display the fetal movement data is to place markers on a graphical user interface representing times at which fetal movement is detected. The markers may be a text, marker, line and/or colours/shading. Alternatively, trend lines may be used to plot quantification of a number of fetal movements detected over a set time period such as a 10, 30, or 60-minute period. Another alternative is to used indication markers identifying whether fetal movement rates have changed over time. Examples of indication markers include text, markers, lines and/or colours/shading.

The display of fetal movement may be done during a monitoring session that occurs for a predetermined duration such as 10, 30, or 60 minutes. A comparative display shows sessions that occur at different time points during gestation, such as 32, 33, or 34 weeks. The comparison information may be between earlier readings for the same patient or may compare readings from current patient with readings from one or more other patients. In one example, one or more patients may be selected using maternal presentation information, for example where the maternal presentation information is similar. Alternatively, or in addition, one or more patients may be selected using patient data from the fetal movement monitoring device, for example where the patient data is similar. When using information from multiple patients, an option is to use averaged fetal movement information from multiple patients. The markers may be presented based on time, location or fetal presentation. When the fetal movement markers are time based the markers are shown at time points fetal movement was felt or detected. When the fetal movement markers are location based, the markers may be shown respect to locations on the abdomen, that is, where in the abdomen the fetal movement was felt. For fetal presentation, the fetal movement markers may be shown based on what part of baby is moving.

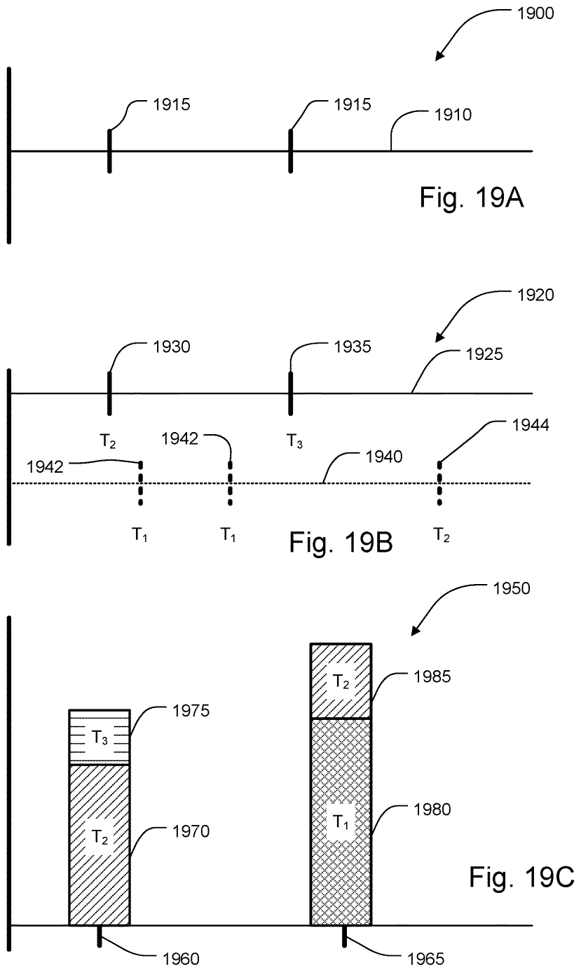

Examples of fetal movement displays will now be described in relation to FIGS. 19A, 19B and 19C. FIG. 19A shows a fetal movement timeline 1900 with a timeline 1910 and fetal movements 1915 indicated on the timeline 1910. The fetal movements 1915 shows the time when the fetal movement was detected. FIG. 19B shows fetal movement comparison timelines 1920 which includes a current timeline 1925 and an earlier timeline 1940. Each of the timelines show not just when fetal movement occurs, but also indicates a fetal movement type. The current timeline 1925 shows a type 1 fetal movement 1930 and a type 2 fetal movement 1935, while the earlier timeline 1940 shows type 1 fetal movements 1942 and a type 2 fetal movement 1944. The earlier timeline 1940 may be for the same patient, a different patient or an average of two or more other patients. When the earlier timeline 1940 shows information from one or more other patients, the samples may be recorded at different or the same gestation age.

FIG. 19C shows a fetal movement chart 1950 that shows an earlier time bar 1960 and a current time bar 1965. The fetal movement chart 1950 is a bar chart displaying a tally of fetal movements for a current session and an earlier session. As discussed above, the earlier session may be for the same patient, a different patient or an average of two or more other patients. The y-axis of the fetal movement chart 1950 is a count of a number of fetal movements detected. The fetal movement chart 1950 is shown as a stacked bar chart, with the earlier time bar 1960 showing a sum of type 2 fetal movement 1970 and a sum of type 3 fetal movement 1975. The current time bar 1965 has a sum of type 1 fetal movement 1980 and a sum of type 2 fetal movement 1985. The fetal movement types shown in FIGS. 19B and 19C may be replaced with the movement name, such as a kick, flutter, swish or roll. The time periods used to generate the two bars may be the same, but may also be different. In one example, two monitoring sessions may be selected with the monitoring sessions having different durations. Alternatively, a predefined window may be selected in each session. In one example, the two timelines may be displayed at the same scale, with the shorter duration having a shorter timeline.

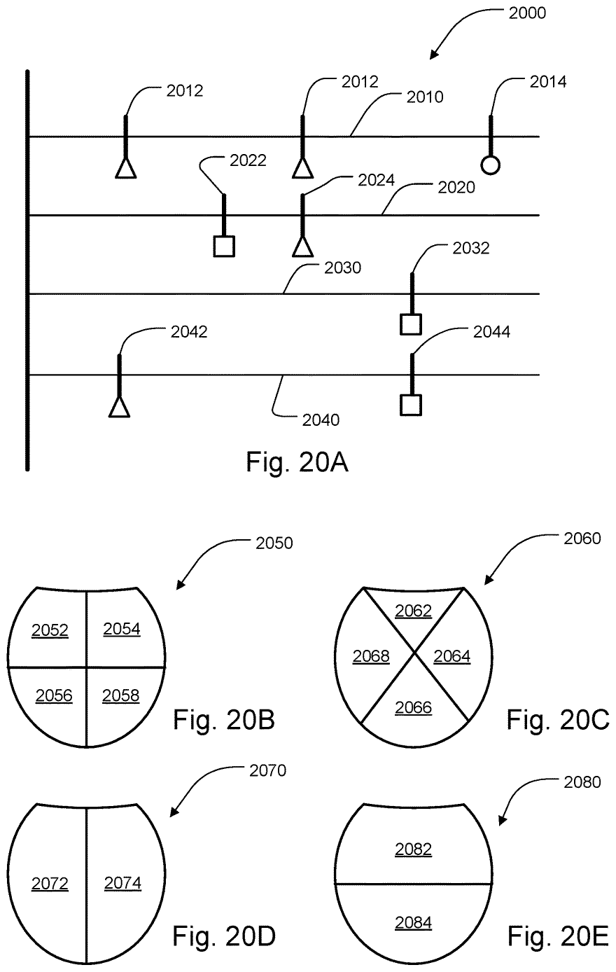

FIG. 20A shows fetal movement zone timelines 2000 which shows four timelines, zone 1 fetal movement timeline 2010, zone 2 fetal movement timeline 2020, zone 3 fetal movement timeline 2030 and zone 4 fetal movement timeline 2040. The fetal movement shown is has timelines grouped according to fetal movement location, that is, where the fetal movement was recorded on the patient. The fetal movement zone timelines 2000 show different fetal movement types using different markers for each type of movement. The zone 1 fetal movement timeline 2010 shows type 1 fetal movements 2012 and a type 3 fetal movement 2014. The zone 2 fetal movement timeline 2020 shows a type 2 fetal movement 2022 and a type 1 fetal movement 2024. The zone 3 fetal movement timeline 2030 has a single type 2 fetal movement 2032, while the zone 4 fetal movement timeline 2040 shows a type 1 fetal movement 2042 and a type 2 fetal movement 2044. While symbols represent the different fetal movement types, colour or other indications may also be used. In some instances, the same fetal movement even may be displayed on two or more timelines.

FIGS. 20B, 20C, 20D and 20E show examples of different fetal movement zones on a patient. FIG. 20B shows fetal movement zones 2050 on an abdomen, with a upper left fetal movement zone 2052, upper right fetal movement zone 2054, lower left fetal movement zone 2056 and lower right fetal movement zone 2058. FIG. 20C shows fetal movement zones 2060 on an abdomen with an upper fetal movement zone 2062, right fetal movement zone 2064, lower fetal movement zone 2066 and left fetal movement zone 2068. FIG. 20C shows fetal movement zones 2070 on an abdomen, with a left fetal movement zone 2072 and a right fetal movement zone 2074. FIG. 20E shows fetal movement zones 2080 on an abdomen with a top fetal movement zone 2082 and a bottom fetal movement zone 2084. While the above examples show the abdomen divided in to two or four zones, other numbers of zones and zone shapes may also be used. The fetal movement zones 2050 or the fetal movement zones 2060 may represent each of the zone 1 fetal movement timeline 2010, the zone 2 fetal movement timeline 2020, the zone 3 fetal movement timeline 2030 and the zone 4 fetal movement timeline 2040 of the fetal movement zone timelines 2000.

In one example, the fetal movement may be shown as a count of movement detected in each zone of FIGS. 20B, 20C, 20D and 20E. The count may be displayed using a numeric display, a heat zone or other type of display.

### Variations

In the above description a nonlinear model is used. Examples of nonlinear models that may be used include support vector machines, Gaussian processes, decision trees, graphical models, and neural networks such as recurrent neural networks, multilayer perceptrons, and convolutional neural networks. The nonlinear models may be trained using known techniques.

The sensors used in the fetal movement monitoring device, as described above, include EMG with electrodes, flex sensor, temperature and accelerometer, however features measured by these sensor may be measure, to a varying degree, by alternative sensors. For example, the signals from an electrode are electrical potentials at multiple points. Equivalent information may be gained from an ECG or EHG which also include cardiac and uterine activity. The signals from a flex sensor are deformation at multiple points. An alternative to a flex sensor may be a stretch sensor. The signals from an accelerometer can be converted to velocity and displacement. An alternative sensor that provides the same information may be a gyro. The signals from a temperature sensor may be used to measure a changes in effort/maternal response to events such as contractions. Alternative sensors may be used to quantify presence of something like sweat that may be correlated to these events such as contractions, in addition to temperature fluctuations.

The sliding windows described in EMG fetal movement monitoring process 900, processing system 100, accelerometer fetal movement monitoring process 1100 and alternative multi-sensor fetal movement monitoring process 1300 process form part of a process that uses patient data to determine a measure of fetal movement by applying a sliding window to the patient data.

Where display of fetal movement is described above, such information may, when suitable, be indicated to a user of the fetal movement monitoring device as an audible warning and/or using a visual display. In one example, an audible notification may be sounded when fetal movement is detected. In another example, different notifications may be used to signal different fetal movement types. Other information may also be indicated to the user.

### Results

A pilot study was conducted during the antepartum stage (36+ weeks gestation) where a wearable device, such as the medical device 500 described above, was attached to a patient in parallel to a cardiotocograph (CTG) which is a type of electronic fetal monitoring. The CTG contains a push button that is pressed by the subject each time fetal movement is felt and the output was recorded as an indicator marker on a printed graph as well as maternal heart rate, fetal heart rate and uterine activity. Such a test allowed comparison to the current standard of care. A total of 7 subjects were randomly selected and a trained clinical midwife identified the times at which these indicator markers were present for times varying between 10 and 30 minutes, with a minimum of 7 and maximum of 20 annotations. The sensor data and CTG data were time synchronised and the Positive Percent Agreement (PPA) was calculated to compare the fetal movements identified by the fetal movement monitoring processes and those identified by the patient. The PPA was calculated according to:

$$( \frac{\#true positives}{\#true positives + \# false negative} )$$

A summary of initial results utilising 83 annotations of fetal movements is shown in table 1. Case 1 was performed using EMG fetal movement monitoring process 900. Case 2 was performed using the flex sensor fetal movement monitoring process 1000, while Case 3 was performed using an EMG and a flex sensor according to the multi-sensor fetal movement monitoring process 1200.

TABLE 1
Initial results summary

| Method | PPA |
|---|---|
| Case 1 - EMG only | 79% |
| Case 2 - Flex only | 80% |
| Case 4 - EMG + Flex | 90% |

The results show that accuracy of the fetal movement detection improves using a multi-sensor approach.

### Advantages and Interpretation

The described fetal movement monitoring processes provides a means for determining fetal movement without the active participation of the pregnant individual. The fetal movement monitoring device provides a simple to use device to determine fetal movement that can provide clinicians with accurate fetal movement detection.

The current standard of care for detection of fetal movement provides only an ability to mark that fetal movement has occurred. The patient can press a button when they feel fetal movement and a marker is placed on a chart. Such an arrangement provides no further data about a type, location or intensity of the fetal movement that may be determined using the above described systems and processes.

As used herein, the term “set” corresponds to or is defined as a non-empty finite organization of elements that mathematically exhibits a cardinality of at least 1 (i.e., a set as defined herein can correspond to a unit, singlet, or single element set, or a multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). Thus, a set includes at least one element. In general, an element of a set can include or be one or more portions of a system, an apparatus, a device, a structure, an object, a process, a procedure, physical parameter, or a value depending upon the type of set under consideration.

The drawings included herewith show aspects of non-limiting representative embodiments in accordance with the present disclosure, and particular structural elements shown in the drawings may not be shown to scale or precisely to scale relative to each other. The depiction of a given element or consideration or use of a particular element number in a particular figure or a reference thereto in corresponding descriptive material can encompass the same, an equivalent, an analogous, categorically analogous, or similar element or element number identified in another drawing or descriptive material associated therewith. The presence of “/” in a drawing or text herein is understood to mean “and/or” unless otherwise indicated, i.e., “A/B” is understood to mean “A” or “B” or “A and B”. The recitation of a particular numerical value or value range herein is understood to include or be a recitation of an approximate numerical value or value range, for instance, within +/−20%, +/−15%, +/−10%, +/−5%, +/−2.5%, +/−2%, +/−1%, +/−0.5%, or +/−0%. The term “essentially all” or “substantially” can indicate a percentage greater than or equal to 50%, 60%, 70%, 80%, or 90%, for instance, 92.5%, 95%, 97.5%, 99%, or 100%.

Many modifications will be apparent to those skilled in the art without departing from the scope of the present invention.

The reference in this specification to any prior publication (or information derived from the prior publication), or to any matter which is known, is not, and should not be taken as an acknowledgment or admission or any form of suggestion that the prior publication (or information derived from the prior publication) or known matter forms part of the common general knowledge in the field of endeavour to which this specification relates.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

1. A method of determining fetal movement during pregnancy or labour, the method comprising:
receiving patient data from measurements from a first sensor and a second sensor attached to a patient, each of the first and second sensors being selected from a set of sensor types consisting of an EMG (Electromyography) sensor, a flex sensor and an accelerometer, the patient data from the second sensor allowing detection of fetal movement when physiological signals impede detection of the fetal movement in the patient data from the first sensor,
wherein the first sensor is a different sensor type from the second sensor; and
determining that fetal movement has occurred by processing the received patient data from the first and second sensors in order to indicate the determined fetal movement.

2. The method according to claim 1, wherein the patient data from the first sensor and the second sensor are processed separately to determine that fetal movement has occurred and the determination of fetal movement based on the first sensor and the second sensor are combined to determine fetal movement.

3. The method according to claim 1, wherein the patient data from the first sensor and the second sensor are used as input to a trained classifier to determine that fetal movement has occurred and the patient data from the first sensor and the second sensor are used to generate a feature vector that is input to the trained classifier.

4. (canceled)

5. (canceled)

6. The method according to claim 1, wherein the first sensor is an accelerometer and processing the patient data to determine a measure of fetal movement comprises determining a displacement.

7. (canceled)

8. (canceled)

9. (canceled)

10. The method according to claim 1, wherein the indication of the determined fetal movement includes a location based display providing an indication where on the patient the fetal movement was detected.

11. The method according to claim 1, wherein the first sensor is an EMG sensor attached to the patient and determining a location of the fetal movement comprises:
determining electrode pair vectors between individual electrodes of the EMG sensor;
selecting two of the electrode pair vectors having a similar signal;
determining the location of the fetal movement by selecting an electrode from the electrodes of the EMG sensor, the selected electrode being common to the selected two electrode pair vectors and positioned at the determined location.

12. (canceled)

13. A method of determining fetal movement during pregnancy or labour, the method comprising:
receiving patient data from measurements from a first sensor and a second sensor attached to a patient, each of the first and second sensors being selected from a set of sensor types consisting of an EMG (Electromyography) sensor, a flex sensor and an accelerometer; and
determining a type of fetal movement that occurred by processing the received patient data from the first and second sensors in order to indicate the type of fetal movement, the patient data from the first sensor allowing determination of a type of fetal movement in combination with the patient data from the second sensor.

14. (canceled)

15. The method according to claim 13, wherein the fetal movement type is determined according to an intensity of the fetal movement.

16. (canceled)

17. The method according to claim 15, wherein the first sensor is an EMG sensor attached to the patient and determining a location of the fetal movement comprises:
determining electrode pair vectors between individual electrodes of the EMG sensor;
selecting two of the electrode pair vectors having a similar signal;
determining the location of the fetal movement by selecting an electrode from the electrodes of the EMG sensor, the selected electrode being common to the selected two electrode pair vectors and positioned at the determined location.

18. The method according to claim 17, wherein fetal movement is determined to occur at a second electrode of the electrodes of the EMG sensor, based on the electrode pair vectors, the second electrode being attached to the patient at a second location.

19. (canceled)

20. The method according to claim 17, wherein the second sensor is a flex senor and a location of the flex sensor are used to determine the location of the fetal movement.

21. The method according to claim 15, wherein one of the first and second sensors is an accelerometer.

22. The method according to claim 21, wherein the patient data from the accelerometer is acceleration data allowing differentiation of the intensity of the fetal movement between small and large movement.

23. The method according to claim 15, wherein the intensity of the fetal movement is determined according to the combination of the patient data from the first and the second sensors.

24. (canceled)

25. The method according to claim 13, wherein respective sensor types of the first sensor and the second sensor are used to determine the fetal movement type.

26. (canceled)

27. The method according to claim 13, further comprising:
receiving maternal input of fetal movement perception and comparing the maternal input with the determined fetal movement.

28. The method according to claim 13, wherein determining the fetal movement uses the maternal presentation information.

29. The method according to claim 13, wherein the indicating of the type of fetal movement includes a time based display showing a time when the fetal movement was detected.

30. The method according to claim 13, wherein the indicating of the type of fetal movement includes a location based display providing an indication where on the patient the fetal movement was detected.

31. (canceled)

32. (canceled)

33. A system configured to perform a method of determining fetal movement during pregnancy or labour, the method comprising:
receiving patient data from measurements from a first sensor and a second sensor attached to a patient, each of the first and second sensors being selected from the set of sensor types consisting of an EMG (Electromyography) sensor, a flex sensor and an accelerometer; and
determining a type of fetal movement that occurred by processing the received patient data from the first and second sensors in order to indicate the type of fetal movement, the patient data from the first sensor allowing determination of a type of fetal movement in combination with the patient data from the second sensor.
